(19) United States
(12) Patent Application Publication
(10) Pub. No.: US 2012/0058901 A1
(43) Pub. Date: Mar. 8, 2012

(54) ASSAY FOR THE DETECTION OF RECURRENCE IN BREAST CANCER USING THE NOVEL TUMOR SUPPRESSOR DEAR1

(75) Inventors: Ann M. Killary, Houston, TX (US); Steven T. Lott, Houston, TX (US); Bruce G. Haffty, Somerset, NJ (US)

(73) Assignee: The Board of Regents of the University of Texas System

(21) Appl. No.: 13/201,296

(22) PCT Filed: Feb. 12, 2010
(86) PCT No.: PCT/US10/24080

§ 371 Date: Oct. 28, 2011

Related U.S. Application Data
(60) Provisional application No. 61/152,334, filed on Feb. 13, 2009.

(51) Int. Cl. C40B 30/00; G01N 33/566; G01N 33/559; G01N 21/76; G01N 33/577; G01N 27/447; C12Q 1/68; G01N 21/64

(52) U.S. Cl. 506/7; 435/6.11; 435/6.12; 435/7.92; 435/7.1; 204/456; 204/464

(57) ABSTRACT

Certain aspects of the present invention relate to new diagnostic and prognostic methods involving DEAR1, a gene is located on the short arm of human chromosome 1. Specifically, analysis of expression or structure of this gene for prognosis or recurrence risk assessment is disclosed.

This application claims priority to U.S. Application No. 61/152,334 filed on Feb. 13, 2009, the entire disclosure of which is specifically incorporated herein by reference in its entirety without disclaimer.

This invention was made with government support by the U.S. Department of Defense DAMD17-02-1-0453-1 and the U.S. National Cancer Institute Early Detection Research Network CA111202-05. The government has certain rights in the invention.

### BACKGROUND OF THE INVENTION

I. Field of the Invention

The present invention relates to the fields of oncology, genetics and molecular biology. More particularly the invention relates to diagnosis and prognosis of cancer, specifically, breast cancer.

II. Description of Related Art

One of the most important factors in the survival of cancer is detection at an early stage. Clinical assays that detect the early events of cancer offer an opportunity to intervene and prevent cancer progression. With the development of gene profiling and proteomics there has been significant progress in the identification of molecular markers or “biomarkers” that can be used to diagnose specific cancers. For example, in the case of prostate cancer, the antigen PSA (prostate specific antigen) can be detected in the blood and is indicative of the presence of prostate cancer. Thus, the blood of men at risk for prostate cancer can be quickly, easily, and safely screened for elevated PSA levels.

Even though there has been significant progress in the field of cancer detection, there still remains a need in the art for the identification of new biomarkers for a variety of cancers that can be easily used in clinical applications. For example, to date there are relatively few options available for the diagnosis of breast cancer using easily detectable biomarkers. The identification of cancer biomarkers is particularly relevant to improving diagnosis, prognosis, and treatment of the disease. As such, there is need in the art to identify alternative biomarkers that can be quickly, easily, and safely detected. Such biomarkers may be used to diagnose, to stage, or to monitor the progression or treatment of a subject with cancer, such as breast cancer, in particular, an invasive, potentially metastatic stage of the disease.

Cancer treatment, such as chemotherapy, radiation and/or surgery, has associated risks, and it would be useful to be able to optimally select patients most likely to benefit. Prognostic testing is useful, for example, to identify patients with poor prognoses such that a more aggressive, higher risk treatment approach is appropriate, and to identify patients with good prognoses for whom risky therapy would not provide enough benefit to warrant the risks. There is an urgent need for new cancer prognostic factors.

### SUMMARY OF THE INVENTION

Certain aspects of the present invention relate to the use of a recently identified tumor suppressor DEAR1 (Ductal Epithelium Associated Ring Chromosome 1, also annotated as TRIM62), encoded by a gene in the 1p35.1 locus (originally the inventors mapped DEAR1 to 1p32 and later localized the gene to 1p35.1 with refinements to the physical map, as demonstrated in Example 1). Certain aspects of the invention are based, in part, on the discovery that alterations in DEAR1 function show a strong predictive value for future risk of aggressive disease and outcome. For example, it might be possible to use DEAR1 expression and/or function assessment to identify women with early-onset breast cancer who have an increased risk of local recurrence so that they get the most appropriate treatment for their cancer.

Therefore, in certain aspects, there may be provided a method of evaluating prognosis of a subject having cancer. Such a method may comprise determining if a cancer cell of the subject has a reduced level of DEAR1 expression and/or function as compared to a reference level, wherein a reduced level of DEAR1 expression and/or function indicates a poor prognosis. The method may be performed in vitro or in vivo. In a further aspect, a level of DEAR1 expression and/or function in the cancer cell of the subject comparable to the reference level indicates a favorable prognosis. In certain aspects, the method may further comprise reporting the prognosis evaluation. For example, the report may be stored in a computer readable media.

In a further aspect, determining if the cancer cell has a reduced level of DEAR1 expression comprises determining the level of DEAR1 expression. In one aspect, DEAR1 mRNA expression may be evaluated to determine the level of DEAR1 expression. In other aspects, DEAR1 protein expression may be evaluated.

In other aspects, determining if the cancer cell has a reduced level of DEAR1 function comprises identifying the DEAR1 gene structure in the cancer cell. For example, if the DEAR1 gene structure has a loss-of-of function mutation or deletion, the cancer cell is determined to have a reduced level of DEAR1 function.

In some aspects, determining if the cancer cell has a reduced level of DEAR1 expression and/or function comprises evaluating a report comprising the DEAR1 expression and/or function information. For example, such a report may be available by specialized service providers on expression profiling or nucleic acid sequence analysis.

As used herein, a “prognosis” generally refers to a forecast or prediction of the probable course or outcome of the cancer. For example, the prognosis may includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, likelihood of metastasis in a patient susceptible to or diagnosed with a cancer, and/or response to a conventional cancer treatment. In a particular aspect, the poor prognosis comprises a high risk of cancer recurrence. The cancer recurrence may be a local recurrence or a distal recurrence.

As used herein, “high risk of recurrence” may mean a lower chance of recurrence-free survival in about 5 years, 10 years or 20 years or any intermitting time range after treatment than mean recurrence-free survival. As used herein, “low risk of recurrence” may mean a higher chance of recurrence-free survival in about 5 years, 10 years or 20 years or any intermitting time range after treatment than mean recurrence-free survival.

In certain aspects, the method may further comprise making or recommending a treatment or a post-treatment follow-up plan based on the determination of DEAR1 expression and/or function. For example, the method may further comprise stratifying subject based on the determination of the DEAR1 function/expression level. The method may further comprise developing a treatment plan based on the determination of the DEAR1 function/expression level. For example, the method may comprise treating a subject determined to have a reduced DEAR1 function/expression level compared with a reference level with a targeted therapy.

These methods may help make an informed decision on treatment options, depending on during which stage it is carried at. The subject for sample collection may be in remission, or before, during or after treatment. In a particular aspect, the subject may be undergoing cancer treatment. The methods help determine if more aggressive surveillance and/or treatment might be needed. The sample may be obtained at the time of a treatment (e.g., surgery) to evaluate expression/mutation, after, or prior to treatment. In particularly embodiments, such a sample may be a fluid sample such as a potentially fine needle aspiration biopsy sample, or a solid sample such as a tumor resection sample. In a further embodiment, the sample may be used for determining expression and/or mutation so as to make prognosis and/or assess the risk of recurrence. In certain aspects, determining the risk of recurrence prior to surgery might influence whether the patient elects to have a treatment such as lumpectomy or mastectomy; determining the risk at the time of surgery might indicate that the individual is at an increased risk for recurrence and would necessitate increased vigilance for follow-up. The treatment may be surgery, radiotherapy, chemotherapy, and/or immunotherapy.

DEAR1 function may be evaluated by gene structure and/or expression level, and may be important for protecting cancer patients from relapse or for treatment outcome. Its function is considered to be a critical regulator of the cellular architecture of large protein complexes associated with development, differentiation and oncogenesis. As shown in the Examples, introduction of DEAR1 wild-type could complement a mutation by initiating acinar morphogenesis and restore normal acinar structure in the mammary gland. Inactivation of a gene upstream to DEAR1 or a positive regulator of DEAR1 function, and/or over-expression of a negative regulator of DEAR1 function may also have effects in DEAR1 function.

“Reduced level of DEAR1 function” or “reduced level of DEAR1 expression” refers to the absence or reduced expression and/or function of DEAR1 function relative to a reference level.

The reference level is a reference level of expression and/or function from non-cancerous tissue from the same subject. Alternatively, the reference level may be a reference level of expression and/or function from a different subject or group of subjects. For example, the reference level of expression and/or function may be an expression and/or function level obtained from tissue of a subject or group of subjects without cancer, an expression level obtained from tissue of a subject or group of subjects with cancer known to have a poor prognosis for survival, or the expression from tissue of a subject or group of subjects with cancer that are known to have a good prognosis. The reference level may be a single value or may be a range of values. The reference level of expression can be determined using any method known to those of ordinary skill in the art. In some embodiments, the reference level is an average level of expression and/or function determined from a cohort of subjects with cancer. The reference level may also be depicted graphically as an area on a graph.

The functional assessment can involve evaluating the structure of the DEAR1 gene, such as an assay selected from the group consisting of sequencing, wild-type oligonucleotide hybridization, mutant oligonucleotide hybridization, SSCP, PCR and RNase protection, or an assay determining loss of heterozygosity, promoter mutation or DNA methylation. Normal gene structure of DEAR1 is indicative of a favorable prognosis and/or a low risk of recurrence. “DEAR1 normal gene structure” is defined as a DEAR1 gene structure that provides a functional DEAR1. A loss-of-function mutated gene structure is indicative of a poor prognosis and/or a high risk of recurrence.

The reduction in function or loss-of-function may result in absence or reduced protein expression or expression of non-functional proteins. In certain aspects, the non-functional proteins may be caused by mutations. In some embodiments, the mutation may be nonsense mutations, dominant negative mutations or missense mutations. In particular embodiments, such a loss-of-function mutated gene structure may encode a mutated protein having one or more mutations at amino acid position 187 or 473, like R187W, R187Q or V473I, or any mutations that reduce the function of DEAR1 in a cell. In further aspects, the loss-of-function mutated gene structure may result in a homologous deletion, loss of heterozygosity or a promoter mutation.

In a further aspect, the assessment can involve assaying the expression of DEAR1. A detectable level of DEAR1 may be indicative of a favorable prognosis and/or a low risk of recurrence, and a lack of detectable level of DEAR1 may be indicative of a poor prognosis and/or a high risk of recurrence. “A detectable level” refers to a level that can be detected by any conventional methods for gene expression known in the art, for example, immunohistochemistry.

This expression assay step may comprise assaying for a DEAR1 transcript (i.e., RNA) expression, for example, Northern blotting, RT-PCR, nuclease protection, an in situ hybridization assay, a chip-based expression platform, invader RNA Assay platform, b-DNA detection platform or any method known in the art. In another aspect, such assessing may comprise contacting the sample with an antibody that binds immunologically to a DEAR1 polypeptide. In certain embodiments, the assessing may comprise, for example, an ELISA, an immunoassay, a radioimmunoassay (RIA), an immunoradiometric assay, a fluoroimmunoassay, a chemiluminescent assay, a bioluminescent assay, a gel electrophoresis, a Western blot analysis, immunohistochemistry, a Luminex® protein assay or any high throughput expression assay in the art. In a particular embodiment, immunological methods such as immunohistochemistry may be extensively used in routine labs to offer a DEAR1-based prognosis and/or recurrence risk assessment for cancer patients undergoing therapy such as resection and subsequent radiotherapy. The antibody used in certain aspects of the present invention may be a polyclonal antibody, a monoclonal antibody or any antigen-binding fragment.

In certain aspects, the subject may have or be suspected of having a cancer of brain, lung, liver, spleen, kidney, lymph node, small intestine, pancreas, blood cells, colon, stomach, breast, endometrium, prostate, testicle, ovary, skin, head and neck, esophagus, bone marrow or blood. Particularly, it may be breast cancer. The cancer may also be an inherited cancer, such as an inherited breast cancer. In further embodiments, the breast cancer may be an early onset breast cancer or metastatic breast cancer. In a further aspect, the sample tested in the invention may be a tissue or fluid sample, such as a sample from surgical resection or a needle aspirate.

Certain embodiments also include a kit comprising a DEAR1 antibody or probes for detecting DEAR1 protein or transcript expression level in a tumor sample and/or a plurality of probes for determining a DEAR1 gene or transcript structure. The kit may be used to determine whether tumor cells comprise a functional or a loss of function of DEAR1 for prediction of recurrence and/or prognosis. The kit may optionally comprise instructions for assessing the results as describe above.

In a further aspect, the kit may comprise instructions for or the method may further comprise stratifying patients for therapeutic options, or developing a treatment plan based on the DEAR1 expression and/function level. Certain aspects of the invention contemplate that a DEAR1 expression and/or function level is indicative of the status of a DEAR1-related pathway and may be used to develop targeted therapy for the DEAR1-related pathway. For example, DEAR1 loss of expression correlates with the triple negative phenotype (clinically negative for expression of estrogen and progesterone receptors (ER/PR) and HER2 protein). Thus the expression/function level of DEAR1 could indicate which genetic pathways are intact and could be targeted for therapies for triple negative cancers. For example, the reduced function and/or expression of DEAR1 may indicate a treatment option for targeting a triple negative disease by using targeted agents, including, but are not limited to, epidermal growth factor receptor (EGFR), vascular endothelial growth factor (VEGF), and poly (ADP-ribose) polymerase (PARP) inhibitors.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan; however, these terms may be used interchangeably with “comprise” or “comprising,” respectively.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A-G. DEAR1 Structure, Mapping and Expression in Normal Tissues. FIG. 1A. Chromosomal localization of DEAR1 as determined by FISH analysis using the DEAR1 P1-derived artificial chromosome (PAC) clone. FIG. 1B. Graphical representation of DEAR1 exonic and protein structure. FIG. 1C. DEAR1 multiple tissue Northern analysis detects a predominant 4.4 kb band in all tissues examined. Additional, lower molecular weight bands were observed in a number of tissues, including heart, placenta, skeletal muscle and brain; FIG. 1D. DEAR1 peptide competition with 5× peptide specifically detects the predicted 54 kD full-length protein in the immortalized HMEC line 76N-E6. FIG. 1E. Transient transfection of HA tagged-DEAR1 into 293T cells (which do not express endogenous DEAR1) detects the appropriate sized protein. FIG. 1F. Western blot analysis of normal tissue protein lysates using the α-N DEAR1 antibody identifies a strong band of approximately 54 kD corresponding to the predicted full-length DEAR1 protein molecular weight; FIG. 1G. Localization of DEAR1 protein in normal tissue assessed by immunohistochemistry using the α-N DEAR1 antibody on a multiple tissue microarray. Significant staining (dark brown, identified by arrow) was observed in epithelial cells found in a wide range of tissues, including (i) bladder (ii) gall bladder (iii) kidney (iv) prostate (v) pancreas and (vi) salivary gland.

FIGS. 2A-C. Downregulation of DEAR1 in Breast Cancer Cell Lines and in Transition to DCIS in the Breast Epithelium. FIGS. 2A-B show immunohistochemical staining of two examples from 14 cases for which both normal ductal structures, DCIS and invasive carcinoma from the same individual are located within the same histological section. Normal ducts are indicated by solid arrows and representative foci of DCIS are indicated by an open arrowhead. Immunohistochemical staining using the α-N DEAR1 antibody appears as a dark brown precipitate. FIG. 2A indicates (i) intense staining of DEAR1 in normal mammary ducts; (ii) diffuse, low level staining of DEAR1 observed in this single focus of DCIS. Note the slight increase in DEAR1 staining towards the center of the focus; (iii) diffuse low level staining of DEAR1 observed throughout much of this region composed of invasive carcinoma. FIG. 2B shows intense staining of DEAR1 noted in the normal duct with a dramatic decrease in expression in adjacent foci of DCIS. FIG. 2C. DEAR1 expression on Western blot analysis of HMEC cultures (normal HMEC as well as immortalized HMECs 76N-E6 and 76N-F2v) and breast carcinoma cell lines.

FIGS. 3A-E. Mutation and Microdeletion Analysis of DEAR1. FIG. 3A. direct genomic sequencing identified a codon 187 missense mutation (C-T) in exon 3 in the 21T series but not in the cell line H16N-2 derived from the normal mammary epithelium from the same patient; FIG. 3B. a missense mutation in codon 473 of exon 5 (GTC-ATC, V-I) detected in a breast tumor sample as well as adjacent normal tissue, but not observed in the normal lymph node from this individual, indicative that the sequence alteration in the tumor was a somatic mutation of the DEAR1 sequence; FIG. 3C. Diagram of genomic structure and core promoter and exon 1 of DEAR1 indicating the location of assays and primers by which HD in tumor 9BT was identified (noted by *) as well as those used for deletion mapping in DEAR1 and flanking genes; FIG. 3D. schematic of homozygous deletion in 9BT; FIG. 3E. STS mapping analysis indicates retention of MS1, deletion of MS2 and retention of MS3 in primary tumor sample (9BT).

FIGS. 4A-D. Introduction of DEAR1 Mediates Acinar Morphogenesis in 3-D Culture. 21MT, control 21MT/Δ187, and wild-type transfectant 21MT/J and 21MT/L analyzed (FIG. 4A) by quantitative RT-PCR and (FIG. 4B) in 3-D culture for the percentage of acinar structures; (FIG. 4C) Propidium (red)-staining structures were photographed by confocal microscopy after 11 days in 3-D culture. The lumen can be clearly seen using DIC microscopy shown to the right of the fluorescent image; (FIG. 4D) confocal images of 21MT, 21MT/Δ, and wild-type transfectant 21MT/J and 21MT/L (i) at low magnification (bar=200 μm) illustrating the dramatic size differences in acini from transfectants with and without wild-type DEAR1 and compared with 21MT cells; (ii) after staining with propidium (red), and E-cadherin (green) discriminated the basal orientation of nuclei and expression of E-cadherin at cell-cell contacts in wild-type transfectants structures propagated in 3-D culture as compared with the large, disorganized apolar structures in 21MT and 21MT/Δ cells (bar=100 μm); (iii) introduction of wild-type DEAR1 into 21MT cells resulted in acinar morphogenesis with epithelial cells visible surrounding a lumen illustrated by staining with propidium (blue) which denotes basal orientation of nuclei, basal orientation of α-6-integrin (red) and increase in Caspase 3 (green) staining in luminal structures in wild-type transfectants as opposed to 21MT and 21MT/Δ.

FIGS. 5A-B. DEAR1 is a Dominant Regulator of Acinar Morphogenesis in HMECs. FIG. 5A. western analysis of shRNA control clones (C1 and C2) and shRNA knockdown clones (sh1, sh2 and sh3); FIG. 5B. confocal images of 3 D culture of control clones (C1 and C2) and DEAR1-knockdown clones (sh1, sh2 and sh3) showing representative acinus stained with alpha6-integrin (red), caspase 3 (green) or DAPI (blue) which shows the clear lumen in controls as opposed to shRNA knockdown clones (FIG. 5B i, ii and iii are results at day 16 and iv is at day 22).

FIG. 6. DEAR1 is an Independent Predictor of Local Recurrence Free Survival in Early Onset Breast Cancer. Immunohistochemical staining of an early onset tissue array resulted in a significant correlation between the expression of DEAR1 and the probability of local recurrence free survival (p=0.0334). At 15 years post diagnosis, recurrence free survival in DEAR1 negative patients was 58% compared to 95% in those patients whose tumors were positive for DEAR1 expression.

FIG. 7. DEAR1 is a highly evolutionarily conserved protein. Alignment of the human, mouse, and rat DEAR1 protein sequences demonstrates significant similarity. Amino acid identity is denoted by “*” in the consensus line, a conserved substitution is denoted by “:” and a non-conserved substitution is seen as a blank space.

FIGS. 8A-B. DEAR1 protein stability. FIG. 8A. Effect of MG132 on DEAR1 protein levels in 21MT cells. Lysates from 21MT, 21MT/J, 21MT/L and 21MT/Δ treated with or without MG132 (5 mM) for 24 h were analyzed by immunoblotting. FIG. 8B. DEAR1 is a stable protein. Lysates from 21MT, 21MT/Δ, 21MT/J and 21MT/L cells treated with 50 mg/ml cycloheximide were analyzed by immunoblotting. The p21 control shows loss of stability following the same treatment.

FIG. 9. Effect of DEAR1 on cell proliferation markers in 3-D culture. Top panel: Ki-67 expression in 21MT series. Bottom panel: BrdU incorporation in DEAR1-KD clones and control clones.

FIGS. 10A-B. Effect of DEAR1 on acinar morphogenesis of MCF7. FIG. 10A. DEAR1 expression was detected from cell lysates on Westerns after DEAR1 transient transfection into MCF7. FIG. 10B. Acinar morphogenesis of MCF7 cells transiently expressing DEAR1 compared with vector at day 19.

FIG. 11. Suppression subtractive hybridization cloning of DEAR1. Microcell hybrids were constructed by the introduction of a normal copy of Chromosome 3 or fragments of Chromosome 3p into a renal cell carcinoma (RCC) cell background ((Sanchez et al., 1994; Lott et al., 1998; Lovell et al., 1999; Killary et al., 1992). Microcell hybrids were injected subcutaneously or orthotopically in athymic nude mice. Results indicated that the entire Chromosome 3 suppressed the formation of tumors and that a small centric fragment (3p12-q11) also suppressed tumors; however, a fragment containing a deletion in the 3p12 region (3p12-q24) failed to suppress tumors, mapping a functional tumor suppressor locus to a 4.75 Mb interval within chromosome 3p12. Microcell hybrids were used as starting materials for SSH library construction. DEAR1 was isolated as one of the cDNAs present in the SSH library.

FIGS. 12A-B. FISH mapping of DEAR1. FIG. 10A. Chromosomal localization of DEAR1 as observed by FISH analysis using the DEAR1 P1-derived artificial chromosome (PAC) clone. Strong signal was observed in the distal region of Chromosome 1p. Based on physical mapping, DEAR1 was mapped to the 1p35.1 interval. FIG. 10B. The 420 kb region harboring DEAR1 is shown in the center of the figure with flanking genes identified. As denoted by the bracket on the Chromosome 1 ideogram, the 1p34-35 region has been shown to have high frequency LOH in sporadic breast cancers with poor prognosis as well as familial breast cancers.

### DETAILED DESCRIPTION OF THE PREFERRED EMBODIMENTS

Certain aspects of the invention are based, in part, on the discovery that the diagnostic and prognostic use of DEAR1. Suppression subtractive hybridization identified DEAR1 as a gene mapping into a region of high frequency loss of heterozygosity (LOH) in a number of histologically diverse human cancers within chromosome 1p35.1. In the breast epithelium, DEAR1 expression is limited to the ducal and glandular epithelium and downregulated in transition to ducal carcinoma in situ (DCIS), an early histological stage in breast tumorigenesis. Significantly, DEAR1 missense mutations and homozygous deletion were discovered in breast cancer cell lines and tumor samples. Introduction of the DEAR1 wild-type and not the missense mutant alleles to complement a mutation in a breast cancer cell line, derived from a 36-year old with invasive breast cancer, initiated acinar morphogenesis in three dimensional (3-D) basement membrane culture and restored tissue architecture reminiscent of normal acinar structures in the mammary gland in vivo. Stable knockdown of DEAR1 in immortalized human mammary epithelial cells (HMECs) recapitulated the growth in 3-D culture of breast cancer cell lines containing DEAR1 mutation in that shDEAR1 clones demonstrated disruption of tissue architecture, loss of apical basal polarity, diffuse apoptosis and failure of lumen formation. Furthermore, immunohistochemical staining of a tissue microarray from a cohort of 123 young female breast cancer patients with 20 year follow-up, indicated that in early onset breast cancer, DEAR1 expression serves as an independent predictor of local recurrence-free survival and correlates significantly with strong family history of breast cancer and the triple negative phenotype (ER⁻, PR⁻, HER2⁻) of breast cancers with poor prognosis.

### I. CANCER

Currently, there is no single marker or combination of markers that can effectively be used to calculate a recurrence estimate for cancer, particularly breast cancer. For individuals with estrogen receptor positive/node negative tumors, an FDA approved gene expression based test is offered by Genomic Health (Oncotype Dx) to calculate a recurrence risk estimate. Unfortunately, the utility of this assay is limited due to the specific requirements of the tumor and the excessive cost of the assay. In certain aspects of this invention, reagents and methods are disclosed to offer a low cost and high-throughput alternative that could be rapidly adopted in most pathology laboratories around the country.

A. Definitions

The term “aggressive” or “invasive” with respect to cancer refers to the proclivity of a tumor for expanding beyond its boundaries into adjacent tissue (Darnell, 1990). Invasive cancer can be contrasted with organ-confined cancer wherein the tumor is confined to a particular organ. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located. For example, invasive bladder cancer includes bladder cancer that is invasive into Muscularis Propria and/or Lamina Propria.

The term “metastasis,” as used herein, refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler et al., 1978; Liotta et al., 1988; Nicolson, 1988; and Zetter, 1990). Increased malignant cell motility has been associated with enhanced metastatic potential in animal as well as human tumors (Hosaka et al., 1978 and Haemmerlin et al., 1981).

“Cancer prognosis” generally refers to a forecast or prediction of the probable course or outcome of the cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

A “subject” or “patient” refers to any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls.

“Remission” refers to a period during which symptoms of disease are reduced (partial remission) or disappear (complete remission). With regard to cancer, remission means there is no sign of it on scans or medical examination. “Remission” is used instead of cure regarding cancer because it cannot be confident that there are no cancer cells at all in the body. Thus, the cancer could recur in the future, although there is no sign of it at the time. More specifically, “remission” could mean the tumor-free time period, and is dated from the first, not the last, therapy session. Patients with tumors that recur within one month of treatment ending are considered to have had no remission. Disappearance of all disease is complete remission; reduction in tumor size by more than 50 percent is considered partial remission.

B. Breast Cancer

(i) Overview

Breast cancer is the most common cause of cancer-related death in women with an early onset of the disease (≦45 years of age) (Jemal et al., 2003). Although early onset breast cancer occurs less frequently than in older women, it is often associated with a poorer prognosis. Compared with older women, young women with breast cancer have a decreased overall survival as well as disease-free survival rates and a higher percentage of tumors with pathologic features reflective of aggressive disease (Bonnier et al., 1995; Harold et al., 1998; Zhou and Recht, 2004; de la Rochefordiere et al., 1993; Haffty et al., 2002). In those early onset breast cancers without nodal involvement, approximately one-fourth will recur up to 12 years post-surgery (Haffty et al., 2002). In addition, younger age is recognized as a risk factor for local-regional recurrence and for distant metastases after either breast conservation treatment (BCT) or mastectomy (de la Rochefordiere et al., 1993; Haffty et al., 2002). Biomarkers are urgently needed to discriminate those younger women who will have an increased risk of recurrence and would therefore benefit from heightened surveillance and adjuvant therapy. However, in order to stratify early onset cancers, the genetic mechanisms that underlie breast cancer in young women must first be elucidated.

The initiation and progression of breast cancer is thought to involve not only a disruption of cellular pathways that underlie proliferation, differentiation, and death, but also perturbation of extracellular signaling pathways that influence differentiation and tissue architecture. The architecture of the human mammary gland is an elaborate branched ductal lobular network terminating in individual acinar units composed of an inner layer of polarized, luminal epithelial cells surrounding a hollow lumen and an outer layer of myoepithelial cells separated from the stroma by an intact basement membrane (Fish and Molitoris, 1994). Concomitant with initiation of tumorigenesis, the mammary gland loses tissue polarity and increases cellular proliferation (Reichmann, 1994). Cell growth, differentiation and death in the mammary epithelium are therefore in intricate balance, the regulation of which is at least in part governed by microenvironmental signals from the extracellular matrix (ECM) (Petersen et al., 1992).

Triple-negative breast cancer is a subtype of breast cancer that is clinically negative for expression of estrogen and progesterone receptors (ER/PR) and HER2 protein. It is characterized by its unique molecular profile, aggressive behavior, distinct patterns of metastasis, and lack of targeted therapies. Although not synonymous, the majority of triple-negative breast cancers carry the “basal-like” molecular profile on gene expression arrays. The majority of BRCA1-associated breast cancers are triple-negative and basal-like; the extent to which the BRCA1 pathway contributes to the behavior of sporadic basal-like breast cancers is an area of active research. Epidemiologic studies illustrate a high prevalence of triple-negative breast cancers among younger women and those of African descent. Increasing evidence suggests that the risk factor profile differs between this subtype and the more common luminal subtypes. Although sensitive to chemotherapy, early relapse is common and a predilection for visceral metastasis, including brain metastasis, is seen. Targeted agents, including epidermal growth factor receptor (EGFR), vascular endothelial growth factor (VEGF), and poly (ADP-ribose) polymerase (PARP) inhibitors, are currently in clinical trials and hold promise in the treatment of this aggressive disease.

Experimental modeling of the ECM using three-dimensional (3-D) basement membrane culture has recapitulated the architecture of mammary ductal epithelium in vitro. (Petersen et al., 1992; Gudjonsson et al., 2003; Weaver et al., 2002). Human mammary epithelial cells (HMECs) as well as the immortalized mammary epithelial cell line MCF10A form polarized growth arrested acini in 3-D culture (Weaver et al., 2002; Debnath et al., 2003). In sharp contrast, breast tumor cell lines propagated in 3-D culture form nonpolarized clusters of cells without acinar formation and with limited differentiation (Petersen et al., 1992). Utilization of the 3-D culture system has elucidated the importance of ECM signaling in the control of differentiation as well as in the initiation and progression of breast tumorigenesis (Petersen et al., 1992; Gudjonsson et al., 2003; Weaver et al., 2002; Muthuswamy et al., 2001; Weaver et al., 1997; Wang et al., 1998; Kirshner et al., 2003; Furuta et al., 2005). Manipulation of the extracellular milieu by activation of key ECM signaling pathways has resulted in the loss of differentiation associated with malignant progression (Weaver et al., 2002; Debnath et al., 2003; Muthuswamy et al., 2001). Likewise, partial or complete restoration of acinar formation in breast cancer cell lines grown in 3-D culture has also been documented (Weaver et al., 1997; Wang et al., 1998; Kirshner et al., 2003). Phenotypic restoration of acinar morphogenesis in 3-D culture was observed irrespective of the accumulation of genetic alterations in the tumor cells, suggesting that the differentiation state in the breast epithelium is in a dynamic state, amenable to therapeutic intervention in the case of breast cancer and that the regulation imposed by the ECM is dominant to tumor-specific mutational events in the control of breast cancer progression.

However, RNAi-mediated knockdown of BRCA1 in MCF10A cells has resulted in a failure of acini formation and increase in proliferation in 3-D culture, suggesting that critical genes, mutated in human breast cancer, could function in the dominant regulation of acinar morphogenesis and differentiation in the mammary epithelium (Furuta et al., 2005). Here parts of the invention are based on the characterization of the gene, DEAR1, for which the genetic complementation of a tumor-associated mutation restores acinar morphogenesis in 3-D culture and stable knockdown of which in immortalized HMECs disrupts this differentiation program. Thus, these studies provide strong evidence of DEAR1 as a novel dominant regulator of acinar morphogenesis and significantly, results herein document DEAR1 expression as an independent predictor of local recurrence-free survival in early onset breast cancer. Together, these data define DEAR1 as a critical genetic link between the control of tissue architecture via ECM remodeling and the loss of differentiation associated with breast cancer.

(ii) Traditional Diagnosis

Certain aspects of the present invention provide novel methods for diagnosing breast cancer by assaying DEAR1, which could be used alone or in combination with known breast cancer biomarkers. Specifically, DEAR1 may be used for predicting risk of recurrence in a subject in remission from cancer treatment or for cancer prognosis. In certain aspects, the method might also predict which carcinoma in situ lesions would progress to an invasive disease and which would not. An example of carcinoma in situ lesions may be ductal carcinoma in situ (DCIS). For example, a reduced level of DEAR expression and/or function may indicate a higher chance of progressing to an invasive disease, and a level of DEAR expression and/or function comparable to a reference level may indicate a lower chance of progressing to an invasive disease.

The genetic mechanisms underlying progression from ductal carcinoma in situ (DCIS), the earliest precursor lesion in breast cancer, to invasive disease is not well understood. Despite the rise in the number of detectable DCIS lesions by improved mammography, which accounts for up to 40% of biopsy specimens detected by mammography, there still is no way to accurately determine which cases of DCIS will never progress and remain indolent and which will recur. One estimate is that 50% of breast relapses recur as invasive disease (Lagios, 1990). Thus, there is a critical need for markers of breast relapse and those that predict relapse free survival.

Major and intensive research has been focused on early detection, treatment and prevention. This has included an emphasis on determining the presence of precancerous or cancerous ductal epithelial cells. These cells are analyzed, for example, for cell morphology, for protein markers, for nucleic acid markers, for chromosomal abnormalities, for biochemical markers, and for other characteristic changes that would signal the presence of cancerous or precancerous cells. This has led to various molecular alterations that have been reported in breast cancer, few of which have been well characterized in human clinical breast specimens. Molecular alterations include presence/absence of estrogen and progesterone steroid receptors, HER-2 expression/amplification (Mark et al., 1999), Ki-67 (an antigen that is present in all stages of the cell cycle except G0 and used as a marker for tumor cell proliferation, and prognostic markers (including oncogenes, tumor suppressor genes, and angiogenesis markers) like p53, p27, Cathepsin D, pS2, multi-drug resistance (MDR) gene, and CD31.

Overexpression of EGFR, particularly coupled with down-regulation of the estrogen receptor, is a marker of poor prognosis in breast cancer patients. Other known markers of breast cancer include high levels of M2 pyruvate kinase (M2 PK) in blood (U.S. Pat. No. 6,358,683), high ZNF217 protein levels in blood (WO 98/02539), and differential expression of a newly identified protein in breast cancer, PDEBC, which is useful for diagnosis (U.S. Patent Publication No. 2003/0124543). Cell surface markers such as CEA, CA-125 and HCG are frequently elevated in the serum of patients with locally advanced and metastatic bladder cancer (Izes et al., 2001), and studies involving circulating levels of tumor-related proteins such as matrix metalloproteinase-2 (Gohji et al., 1996), hepatocyte growth factor (Gohji et al., 2000), and tissue polypeptide antigen (Maulard-Durdux et al., 1997) have shown promise. These biomarkers offer alternative methods of diagnosis; however, they are not widely used.

(iii) Treatment

The mainstay of breast cancer treatment is surgery when the tumor is localized, with possible adjuvant hormonal therapy (with tamoxifen or an aromatase inhibitor), chemotherapy, and/or radiotherapy. At present, the treatment recommendations after surgery (adjuvant therapy) follow a pattern. This pattern is subject to change, as every two years, a worldwide conference takes place in St. Gallen, Switzerland, to discuss the actual results of worldwide multi-center studies. Depending on clinical criteria (age, type of cancer, size, metastasis) patients are roughly divided to high risk and low risk cases, with each risk category following different rules for therapy. Treatment possibilities include radiation therapy, chemotherapy, hormone therapy, and immune therapy.

In planning treatment, doctors can also use PCR tests like Oncotype DX or microarray tests like MammaPrint that predict breast cancer recurrence risk based on gene expression. In February 2007, the MammaPrint test became the first breast cancer predictor to win formal approval from the Food and Drug Administration. This is a new gene test to help predict whether women with early-stage breast cancer will relapse in 5 or 10 years, this could help influence how aggressively the initial tumor is treated.

Interstitial laser thermotherapy (ILT) is an innovative method of treating breast cancer in a minimally invasive manner and without the need for surgical removal, and with the absence of any adverse effect on the health and survival of the patient during intermediate follow up.

Radiation treatment is also used to help destroy cancer cells that may linger after surgery. Radiation can reduce the risk of recurrence by 50-66% (½-⅔rds reduction of risk) when delivered in the correct dose.

### II. THE DEAR1 TUMOR SUPPRESSOR

Breast cancer in young women tends to have a natural history of aggressive disease for which rates of recurrence are higher than in breast cancers detected later in life. Little is known about the genetic pathways that underlie early onset breast cancer. Here certain aspects of the invention involve DEAR1 (Ductal Epithelium Associated Ring Chromosome 1), a gene encoding a member of the TRIM (tripartite motif) subfamily of RING finger proteins (annotated as TRIM 62), and provide evidence for its role as a dominant regulator of acinar morphogenesis in the mammary gland and as an independent predictor of local recurrence-free survival in early onset breast cancer.

Some aspects of the present invention relate to the use of a recently identified tumor suppressor, encoded by a gene in the 1p35.1 locus, and designated here DEAR1. Initially, the inventors designated this gene as CAR-1 (Cancer Associated Ring-1), for example, see U.S. Pat. No. 6,943,245. However, the inventors decided to change its name to DEAR-1 to minimize confusion with other genes named CAR-1 prior the identification of this tumor suppressor. DEAR1 is also annotated in GenBank as TRIM62 (tripartite motif-containing 62), denoting its membership in the TRIM family of protein.

U.S. Pat. No. 6,943,245 describes discovery of this novel gene and related methods of therapeutics and diagnostics, and is hereby incorporated by reference. This molecule is capable of suppressing tumor phenotypes in various cancers.

The term tumor suppressor is well-known to those of skill in the art. Examples of other tumors suppressors are p53, Rb and p16, to name a few. While these molecules are structurally distinct, they form a group of functionally-related molecules, of which DEAR1 is a member. The uses in which these other tumor suppressors now are being exploited are equally applicable here.

In addition to the entire DEAR1 molecule, certain aspects of the present invention also relate to fragments of the polypeptide that may or may not retain the tumor suppressing (or other) activity. Fragments, including the N-terminus of the molecule, may be generated by genetic engineering of translation stop sites within the coding region. Alternatively, treatment of the DEAR1 molecule with proteolytic enzymes, known as proteases, can produces a variety of N-terminal, C-terminal and internal fragments. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Features of the Polypeptide

The gene for DEAR1 encodes either a 475 amino acid polypeptide or a 304 amino acid polypeptide, depending on splicing. When the present application refers to the function of DEAR1 or “wild-type” activity, it is meant that the molecule in question has the ability to inhibit the transformation of a cell from a normally regulated state of proliferation to a malignant state, i.e., one associated with any sort of abnormal growth regulation, or to inhibit the transformation of a cell from an abnormal state to a highly malignant state, e.g., to prevent metastasis or invasive tumor growth. Other phenotypes that may be considered to be regulated by the normal DEAR1 gene product are epithelial to mesenchymal transition, production of stem cells, angiogenesis, adhesion, migration, cell-to-cell signaling, cell growth, cell proliferation, density-dependent growth, anchorage-dependent growth and others. Determination of which molecules possess this activity may be achieved using assays familiar to those of skill in the art. For example, transfer of genes encoding DEAR1, or variants thereof, into cells that do not have a functional DEAR1 product, and hence exhibit impaired growth control, will identify, by virtue of growth suppression, those molecules having DEAR1 function.

B. Variants of DEAR1

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

C. Purification of Proteins

It could be desirable to purify DEAR1 or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with certain aspects of the present invention is discussed below.

D. Synthetic Peptides

Certain aspects of the present invention also describe smaller DEAR1-related peptides for use in various embodiments of the present invention, such as generation of an antibody for diagnostic methods of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

E. Antigen Compositions

Certain aspects of the present invention also provide for the use of DEAR1 proteins or peptides as antigens for the immunization of animals relating to the production of antibodies. It is envisioned that either DEAR1, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

### GENERATING ANTIBODIES REACTIVE WITH DEAR1

In another aspect, the present invention the production and/or use of an antibody that is immunoreactive with a DEAR1 molecule of the present invention, or any portion thereof. An antibody can be a polyclonal or a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of certain aspects of the present invention can be used to immunize one or more experimental animals, such as a rabbit or mouse, which will then proceed to produce specific antibodies against the compounds. Polyclonal antisera may be obtained, after allowing time for antibody generation, simply by bleeding the animal and preparing serum samples from the whole blood.

It is proposed that the monoclonal antibodies of certain aspects of the present invention will find useful application in standard immunochemical procedures, such as ELISA and Western blot methods and in immunohistochemical procedures such as tissue staining, as well as in other procedures which may utilize antibodies specific to DEAR1-related antigen epitopes. Additionally, it is proposed that monoclonal antibodies specific to the particular DEAR1 of different species may be utilized in other useful applications

In general, both polyclonal and monoclonal antibodies against DEAR1 may be used in a variety of embodiments. For example, they may be employed in antibody cloning protocols to obtain cDNAs or genes encoding other DEAR1. They may also be used in inhibition studies to analyze the effects of DEAR1 related peptides in cells or animals. Anti-DEAR1 antibodies will also be useful in immunolocalization studies to analyze the distribution of DEAR1 during various cellular events, for example, to determine the cellular or tissue-specific distribution of DEAR1 polypeptides under different points in the cell cycle. A particularly useful application of such antibodies is in purifying native or recombinant DEAR1, for example, using an antibody affinity column. The operation of all such immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are give in the examples below.

As is well known in the art, a given composition may vary in its immunogenicity. It is often necessary therefore to boost the host immune system, as may be achieved by coupling a peptide or polypeptide immunogen to a carrier. Exemplary carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified DEAR1 protein, polypeptide or peptide or cell expressing high levels of DEAR1. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. MAbs produced by either means may be further purified, if desired, using filtration, centrifugation and various chromatographic methods such as HPLC or affinity chromatography.

### IV. CANCER DIAGNOSIS AND PROGNOSIS INVOLVING DEAR1

Certain aspects of the invention are based on the discovery that DEAR1 expression is a significant marker for local recurrence-free survival until over 20 years postsurgery. Previously, this cohort had been examined for markers that might predict local recurrence, including ER, PR, HER-2/neu, p53 and cytokeratin 19; however, only cytokeratin 19 was statistically significant for predicting local recurrence (Parikh et al., 2008). The finding that DEAR1 independently predicts local recurrence in early onset disease is significant given that local recurrence following breast conservation surgery in younger women is a major clinical issue. Young women with breast cancer have significantly higher rates of local recurrence than older women with local recurrence following breast conservation therapy and radiotherapy occurring earlier and with a worse prognosis in many studies than in older cohorts (Fisher et al., 1991; Fowble et al., 1994; Veronesi et al., 1995; Haffty et al., 1991).

Thus, there is an urgent need to identify prognostic markers to identify women with a heightened risk of recurrence for which more aggressive surveillance and treatment might be warranted, as well as individuals with favorable prognosis, who might be spared rigorous therapeutic regimens and for whom BCT might be the preferred surgical option. It has been demonstrated in certain aspects of the invention that DEAR1 loss of function may play an important role in the loss of differentiation and the poor outcome associated with a high frequency of early onset cancers. The finding that DEAR1 correlates with the triple negative breast cancer subtype also suggests an impact of loss of DEAR1 on the differentiated state in this subtype of basal tumors of the breast. Thus, the clear delineation between DEAR1 expression and recurrence as well as the correlation of DEAR1 expression with the subtype of breast tumors with poor prognosis, suggests that DEAR1 is an important biomarker for stratifying cancer such as an early onset disease; and these data in conjunction with its role as a dominant mediator of differentiation in 3-D culture, would point to a critical role for DEAR1 in a genetic pathway that is important to early onset breast cancer, the elucidation of which could have a significant impact on early detection and targeted therapy for malignancies of the breast.

The tumor suppressor DEAR1 is a gene that certain aspects of the invention have shown to be specifically mutated in breast cancer cell lines and tumor samples, shows loss of expression in transition to ductal carcinoma in situ and when replaced into breast cancer cells to complement a genetic mutation, results in differentiation in SCID mice and restoration of acinar morphogenesis in vitro in 3 dimensional culture. Because several of the mutations in DEAR1 involved early onset breast cancers, a panel of breast cancers from <45 year-old individuals were screened as shown in the Examples for expression of DEAR1. Significantly, DEAR1 shows loss of expression in 61% of the panel understudy and an additional >20% showed only focal expression (i.e., less than 25% of the tumor was positive). Furthermore, positive staining for DEAR1 expression correlated with a >90% relapse free survival as opposed to those tumors with negative DEAR1 staining which showed a significantly poorer relapse free survival (p=0.03). Particularly, some aspects of the invention may provide an antibody based assay to detection expression of DEAR1 protein in tumor samples from surgical resection or fine needle aspirates to help to stratify which breast tumors have a significant chance of relapse versus those with a great than 90% chance of relapse free survival.

DEAR1 and the corresponding gene may be employed as a diagnostic or prognostic indicator of cancer. More specifically, change in expression levels, point mutations, deletions, insertions or regulatory pertubations relating to DEAR1 may cause cancer or promote cancer development, cause or promote tumor progression at a primary site, cause or promote loss of polarity, and/or cause or promote metastasis, specifically, risk of recurrence in remission. Other phenomena associated with malignancy that may be affected by status of DEAR1 expression and/or function include angiogenesis and tissue invasion.

A. Genetic Diagnosis

One embodiment of the instant invention comprises a method for detecting variation in the expression of DEAR1. This may comprises determining the level of DEAR1 or determining specific alterations in the expressed product. This sort of assay has importance in the diagnosis of related cancers. Such cancer may involve cancers of the brain (glioblastomas, medulloblastoma, astrocytoma, oligodendroglioma, ependymomas), lung, liver, spleen, kidney, pancreas, small intestine, blood cells, lymph node, colon, breast, endometrium, stomach, prostate, testicle, ovary, skin, head and neck, esophagus, bone marrow, blood or other tissue. In particular, certain aspects of the present invention relate to the diagnosis of breast cancer.

The biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have DEAR1-related pathologies. In this way, it is possible to correlate the amount or kind of DEAR1 detected with various clinical states.

Various types of defects have been identified by certain aspects of the invention. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of DEAR1 produced, both by altering the transcription of the gene or in destabilizing or otherwise altering the processing of either the transcript (mRNA) or protein.

A cell takes a genetic step toward oncogenic transformation when one allele of a tumor suppressor gene is inactivated due to inheritance of a germline lesion or acquisition of a somatic mutation. The inactivation of the other allele of the gene usually involves a somatic micromutation or chromosomal allelic deletion that results in loss of heterozygosity (LOH). Alternatively, both copies of a tumor suppressor gene may be lost by homozygous deletion.

It is contemplated that other mutations in the DEAR1 gene may be identified in accordance with certain aspects of the present invention, such as one or more epigenetic mutations (for example, change in methylation status), somatic mutations and/or germline mutations. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In particular embodiments, the probes or primers are labeled with radioactive species (³²P, ¹⁴C, ³⁵S, ³H, or other label), with a fluorophore (rhodamine, fluorescein) or a chemiluminescent molecule (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in certain aspects of the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in certain aspects of the present invention, Walker et al. (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with certain aspects of the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with certain aspects of the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, may also be used in the amplification step of certain aspects of the present invention (Wu et al., 1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in certain aspects of the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to certain aspects of the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). Certain aspects of the present invention provide methods by which any or all of these types of analyses may be used. Using the sequences disclosed herein, oligonucleotide primers may be designed to permit the amplification of sequences throughout the DEAR1 gene that may then be analyzed by direct sequencing.

(vi) Kit Components

All the essential materials and reagents required for detecting and sequencing DEAR1 and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe. For example, the kit may be in the format of a microarray, a bead-array, or any high-throughput profiling platform.

(vii) Design and Theoretical Considerations for Relative Quantitative RT-PCR™

Reverse transcription (RT) of RNA to cDNA followed by relative quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR™ assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.

(viii) Chip Technologies

Specifically contemplated by certain aspects of the present invention are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

In certain aspects of the present invention, SNP-based arrays or other gene arrays or chips are also contemplated to determine the presence of wild-type DEAR1 allele and the structure of mutations. A single nucleotide polymorphism (SNP), a variation at a single site in DNA, is the most frequent type of variation in the genome. For example, there are an estimated 5-10 million SNPs in the human genome. As SNPs are highly conserved throughout evolution and within a population, the map of SNPs serves as an excellent genotypic marker for research. An SNP array is a useful tool to study the whole genome.

In addition, SNP array can be used for studying the Loss Of Heterozygosity (LOH). LOH is a form of allelic imbalance that can result from the complete loss of an allele or from an increase in copy number of one allele relative to the other. While other chip-based methods (e.g., comparative genomic hybridization can detect only genomic gains or deletions), SNP array has the additional advantage of detecting copy number neutral LOH due to uniparental disomy (UPD). In UPD, one allele or whole chromosome from one parent are missing leading to reduplication of the other parental allele (uni-parental=from one parent, disomy=duplicated). In a disease setting this occurrence may be pathologic when the wild-type allele (e.g., from the mother) is missing and instead two copies of the heterozygous allele (e.g., from the father) are present. This usage of SNP array has a huge potential in cancer diagnostics as LOH is a prominent characteristic of most human cancers. Recent studies based on the SNP array technology have shown that not only solid tumors (e.g. gastric cancer, liver cancer, etc.) but also hematologic malignancies (ALL, MDS, CML etc) have a high rate of LOH due to genomic deletions or UPD and genomic gains.

(ix) Other High Throughput Gene Expression Platforms

In addition to microarray-based gene expression assays, new platforms for high throughput gene expression platforms have been developed. For example, a mass spectrometric approach for measuring gene expression levels has been developed (Berggren, 2002). This technique utilizes a signal amplification system and analysis by matrix-assisted laser desorption/ionization time-of-flight (MALDI-TOF) mass spectrometry. Signal amplification from the targeted RNA employs a recently developed invasive cleavage assay that does not require prior PCR amplification. The assay uses a set of target-specific probes (oligonucleotides), which hybridize to the RNA being measured to create an overlap structure with a single-stranded flap. This flap is enzymatically cleaved and accumulates linearly in a target-specific manner. The products of the reaction, short DNA oligomers, are well suited for quantitative detection by MALDI-TOF mass spectrometry. Multiplexing is achieved by designing the assays so that reaction products for different mRNA targets have discrete masses that can be resolved in a single mass spectrum.

B. Immunodiagnosis

Certain aspects of the present invention provide methods for diagnosing the recurrence risk of cancer, and in particular breast cancer, by analyzing for expression levels of DEAR1 in cells, tissues or bodily fluids, wherein a detectable level of DEAR1 in the patient is indicative of >90% chance of recurrence-free survival.

Without limiting the instant invention, typically, for a quantitative diagnostic assay a detectable level indicating the patient being tested has breast cancer is one in which cells, tissues, or bodily fluid levels of a cancer marker, such as DEAR1, are at least 1%, 5% or 10% of that in the same cells, tissues, or bodily fluid of a normal human control.

Certain aspects of the present invention also provides a method of diagnosing tumor such as metastatic cancer, and in particular metastatic breast cancer, or a patient having breast cancer which has not yet metastasized. In the method of certain aspects of the present invention, a human cancer patient suspected of having breast cancer which may have metastasized (but which was not previously known to have metastasized) may be identified. This may be accomplished by a variety of means known to those of skill in the art.

In certain aspects of the present invention, determining the presence of DEAR1 in cells, tissues, or bodily fluid, is particularly useful for identifying cancers which have poor prognosis, such as triple negative cancer (ER⁻, PR⁻, HER2⁻) and/or breast cancer associated with a family history. Existing techniques may have difficulty identifying these cancers. However, proper treatment selection is often dependent upon such knowledge.

Normal human control as used herein includes a human patient without cancer and/or non cancerous samples from the patient.

Antibodies of certain aspects of the present invention can be used in characterizing the DEAR1 content of healthy and diseased tissues, through techniques such as immunohistochemistry, ELISAs and Western blotting. This may provide a method for the predicting the risk of recurrence. Particularly, a polyclonal antibody or a monoclonal antibody developed from a DEAR1 protein or portion thereof may be used to screen breast cancer samples to determine to presence or absence of protein expression.

In a particular aspect of the invention, there may also be provided a kit such as an immunohistochemistry kit comprising a plurality of antibodies for determining the DEAR1 expression level and instructions for evaluating prognosis of a subject based on the DEAR1 determination. Immunohistochemistry or IHC refers to the process of localizing proteins in cells of a tissue section exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). IHC is also widely used in basic research to understand the distribution and localization of biomarkers and differentially expressed proteins in different parts of a biological tissue. Visualizing an antibody-antigen interaction can be accomplished in a number of ways. In the most common instance, an antibody is conjugated to an enzyme, such as peroxidase, that can catalyze a colour-producing reaction (see immunoperoxidase staining). Alternatively, the antibody can also be tagged to a fluorophore, such as FITC, rhodamine, Texas Red or Alexa Fluor (see immunofluorescence).

The use of antibodies of certain aspects of the present invention in an ELISA assay is also contemplated. For example, anti-DEAR1 antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for DEAR1 that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

Immunoblot or Western blot analysis may also be use in the present methods. The anti-DEAR1 antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

### V. METHODS OF THERAPY

Certain aspects of the present invention also involves, in another embodiment, the treatment of cancer. It may be contemplated that a wide variety of tumors may be treated in combination with the present DEAR1 diagnostic/prognostic methods, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue. In particular, breast cancer is contemplated.

In certain aspects of the invention, there may be provided a method of treating a subject, comprising treating a subject having a cancer and determined to have a reduced level of DEAR1 expression and/or function with a treatment plan comprising at least one alternative therapy. For example, the alternative therapy may be any therapy other than conventional cancer therapy (including surgery, chemotherapy or radiation therapy), such as angiogenesis inhibitor therapy, immunotherapy, gene therapy, hyperthermia, photodynamic therapy, and/or targeted cancer therapy. In certain aspects, the alternative therapy may be used alone, or in combination with one or more conventional therapies after the determination of DEAR1 status.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved.

A. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of certain aspects of the present invention, chemotherapy, radiotherapy, hormonal therapy, gene therapy, immunotherapy and/or alternative therapies. In certain aspects, the method may further comprising determining the DEAR1 status and developing a treatment plan. For example, if the subject has or is determined to have a reduced DEAR1 expression and/or function level, the subject may have a favorable response to a more aggressive treatment such as mastectomy if the subject has breast cancer. In a further aspect, there may be provided a method comprising treating a breast cancer patient determined to have a reduced DEAR1 expression and/or function level with mastectomy. In other aspects, if the subject has or is determined to have a DEAR1 expression and/or function level comparable to a reference level, the subject may have a favorable response to a less aggressive treatment such as lumpectomy if the subject has breast cancer. In a still further aspect, there may be provided a method comprising treating a breast cancer patient determined to have a DEAR1 expression and/or function level comparable with a reference level with lumpectomy.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that certain aspects of the present invention may be used in conjunction with removal of superficial cancers, precancers, or incidental amounts of normal tissue.

Intratumoral injection prior to surgery or upon excision of part of all of cancerous cells, tissue, or tumor, a cavity may be formed in the body. Treatment may be accomplished by perfusion, direct injection or local application of these areas with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

B. Chemotherapy

A wide variety of chemotherapeutic agents may be used in accordance with certain aspects of the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

(i) Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

(ii) Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

(iii) Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

(iv) Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

(v) Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

(vi) Other Agents

Other agents that may be used include bevacizumab (brand name Avastin®), gefitinib (Iressa®), trastuzumab (Herceptin®), cetuximab (Erbitux®), panitumumab (Vectibix®), bortezomib (Velcade®), and Gleevec. In addition, growth factor inhibitors and small molecule kinase inhibitors have utility in certain aspects of the present invention as well. All therapies described in Cancer: Principles and Practice of Oncology (7^{th} Ed.), 2004, and Clinical Oncology (3^{rd} Ed., 2004) are hereby incorporated by reference. The following additional therapies are encompassed, as well.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with p53 gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of certain aspects of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. In addition, p53 itself may be an immunotherapy target. See U.S. Publication 2005/0171045, incorporated herein by reference

Tumor Necrosis Factor is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

The use of sex hormones according to the methods described herein in the treatment of cancer. While the methods described herein are not limited to the treatment of a specific cancer, this use of hormones has benefits with respect to cancers of the breast, prostate, and endometrial (lining of the uterus). Examples of these hormones are estrogens, anti-estrogens, progesterones, and androgens.

Corticosteroid hormones are useful in treating some types of cancer (lymphoma, leukemias, and multiple myeloma). Corticosteroid hormones can increase the effectiveness of other chemotherapy agents, and consequently, they are frequently used in combination treatments. Prednisone and dexamethasone are examples of corticosteroid hormones.

D. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to certain aspects of the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques. Stereotactic radiotherapy is used to treat brain tumors. This technique directs the radiotherapy from many different angles so that the dose going to the tumor is very high and the dose affecting surrounding healthy tissue is very low. Before treatment, several scans are analyzed by computers to ensure that the radiotherapy is precisely targeted, and the patient's head is held still in a specially made frame while receiving radiotherapy. Several doses are given.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### DEAR1 is a RBBC/TRIM Family Member Mapping into a Region of LOH in Breast Cancer within Chromosome 1p35.1

One of the most studied genomic intervals in human cancer lies within the short arm of human chromosome 1 in which loss of heterozygosity (LOH) within three separate intervals occurs at high frequency in a variety of epithelial cancers, including both sporadic breast cancers and breast cancers with inherited predisposition (Borg et al., 1992; Milikan et al., 1999; Ragnarsso et al., 1999; Reddy et al., 1992). LOH within chromosome 1p has been shown to predict poor prognosis in node negative breast cancers and allelic deletions in the 1p36 and 1p32 region have been found to correlate with poor survival (Reddy et al., 1992).

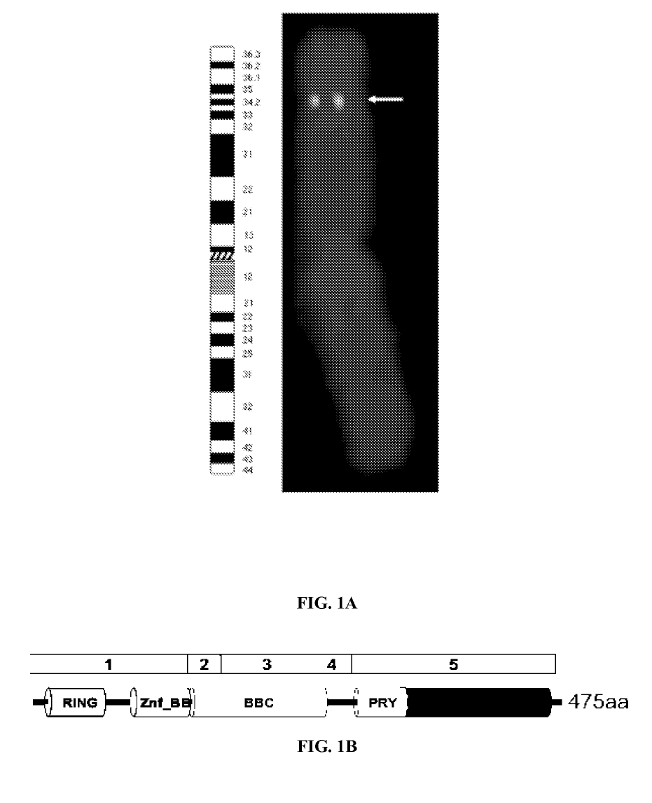

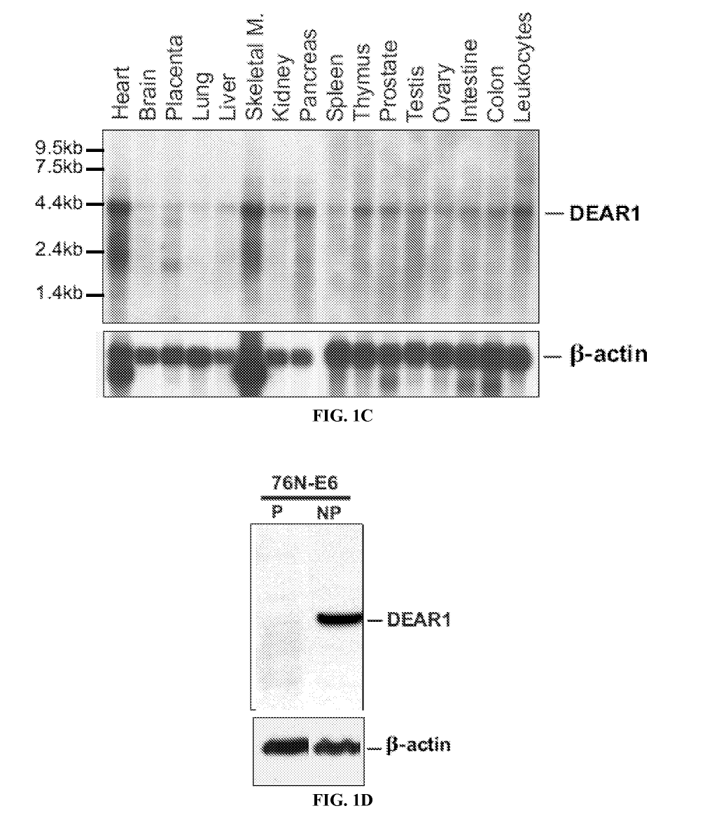

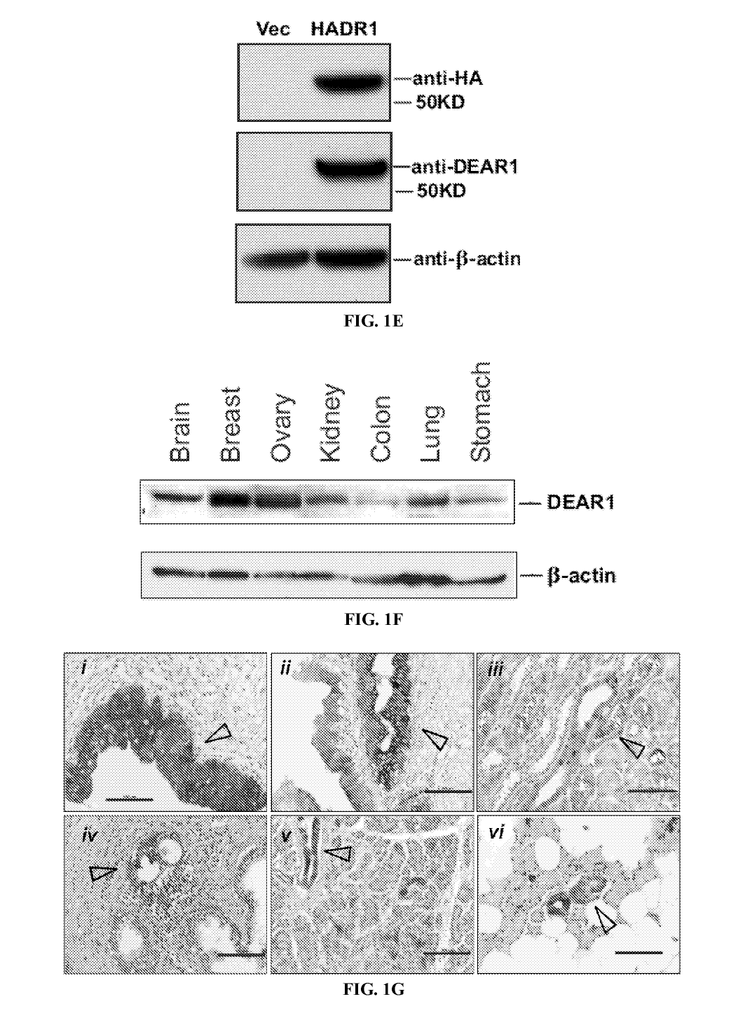

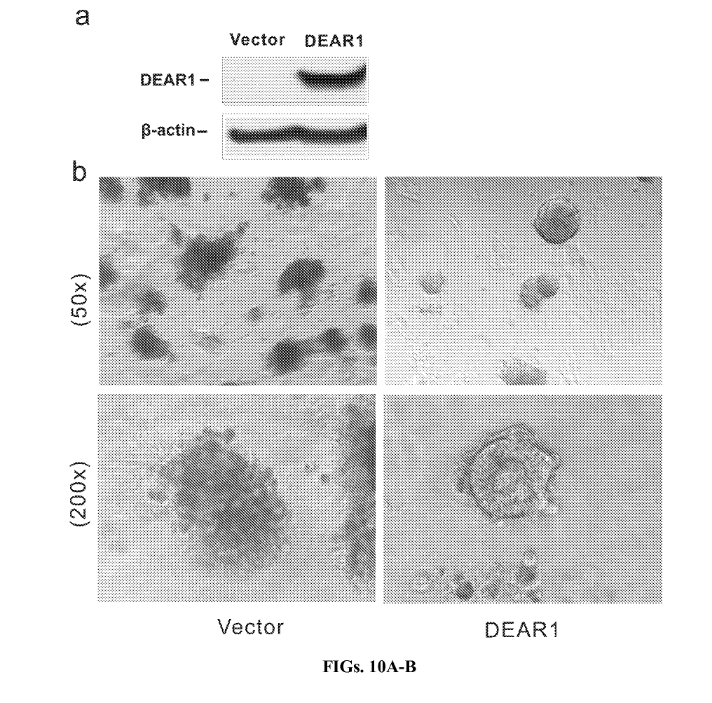

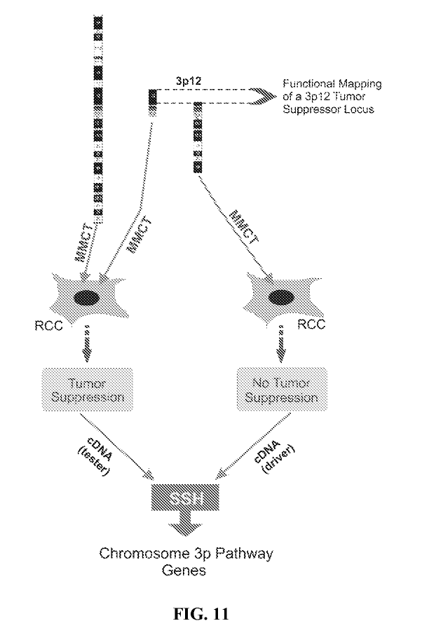

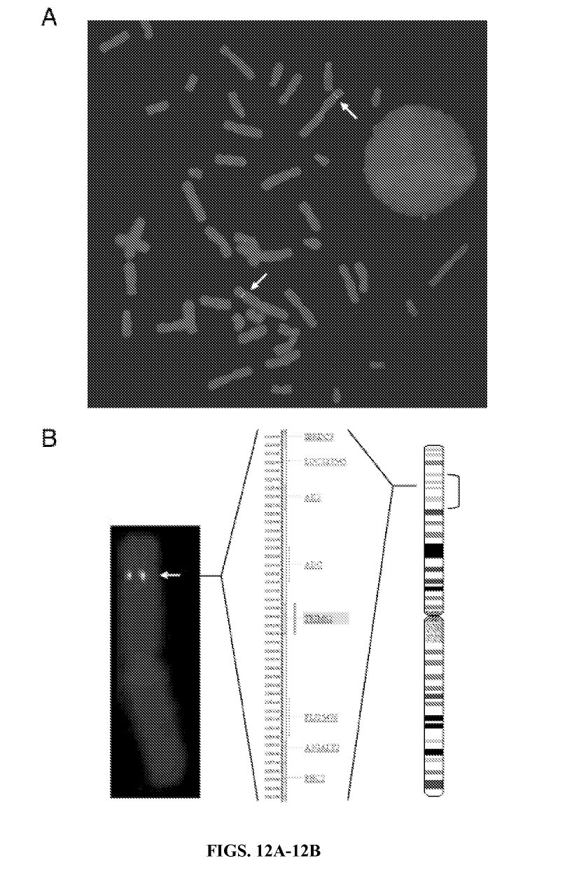

In the screening of cDNAs obtained from a suppression subtractive hybridization library (FIG. 11), a 700 bp cDNA having significant similarity to a RING finger protein (3.9×10⁻¹⁸) was identified that mapped by fluorescence in situ hybridization (FISH) to one of the chromosome 1p genomic intervals with LOH in breast cancer within chromosome 1p34-35. Subsequent screening of the human reference sequence (UCSC version hg18-based on NCBI Build 36 using the BLAT tool on the UCSC Genome Bioinformatics website (available at genome.ucsc.edu) unambiguously mapped the gene, DEAR1 (Ductal Epithelium-associated Ring Chromosome 1), to human chromosome 1p35.1 (FIG. 1A and FIGS. 12A-12B).

The complete DEAR1 open reading frame was identified by sequencing of additional cDNAs obtained from an NT2 neuroepithelial cDNA library screen and reverse transcription PCR of placental RNA. DEAR1 is comprised of 5 exons encoding a 475 amino acid protein with a predicted tripartite sequence motif associated with the RBCC (RING-B-box-Coiled-Coil)/TRIM (tripartite motif) subfamily of RING finger proteins with PRY and SPRY domains present within the carboxy terminus (FIG. 1B) (Freemont, 1993; Borden, 1998; Reymond et al., 2001; Reddy et al., 1991; Kastner et al., 1992; Le Douarin et al., 1995; Urano et al., 2002). The RBCC/TRIM family of RING finger proteins has been shown to play critical roles in the formation and architecture of multiprotein complexes within both the cytoplasm and nucleus, and has been implicated in the regulation of differentiation, development and oncogenesis in multiple cell types and species (Freemont, 1993; Borden, 1998; Reymond et al., 2001). Human family members have been associated with initiating events in oncogenesis, either due to loss of growth/tumor suppressor functions (PML) or gain of oncogenic functions (RFP, Efp) in addition to participating as oncogenic fusion partners in specific chromosomal translocation events, such as PML, TIF1 and RFP (Kastner et al., 1992; Le Douarin et al., 1995; Urano et al., 2002; Isomura et al., 1992). BLAST comparisons to the nonredundant peptide sequence databases indicated that DEAR1 was a unique member of the RBCC/TRIM gene family (hypothetical protein FLJ10759, later annotated as TRIM 62) with the closest similarity to other human RBCC/TRIM family members being a 29% identity with the human Ret finger protein RFP, originally identified as a fusion partner with the RET tyrosine kinase proto-oncogene (Kastner et al., 1992).

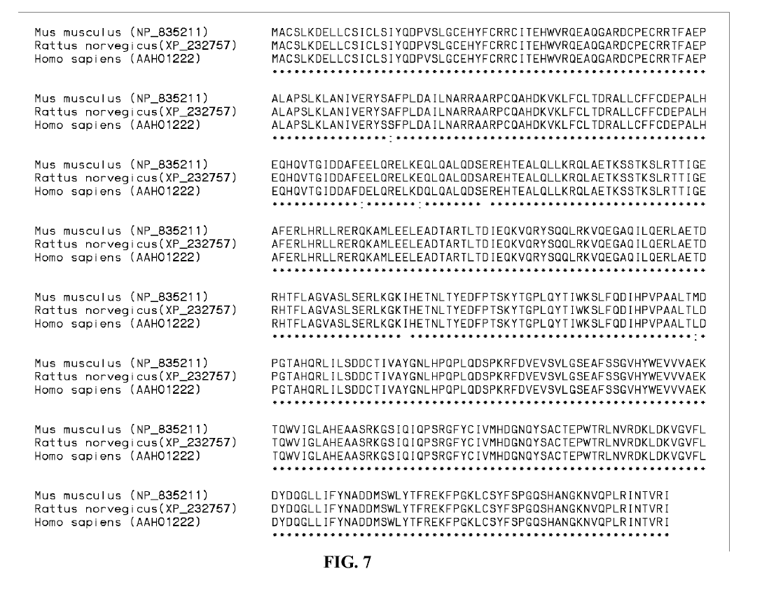

DEAR1 is essentially identical (98%) to mouse and rat sequences (NP_{—}835211 [Mus musculus] and XP_{—}232757 [Rattus norvegicus]) (FIG. 7) as well as RBCC/TRIM proteins from diverse species, including Xenopus laevis XNF7 (33% identity) and TRIM39 (32% identity in mouse, rat and human) (Reddy et al., 1991; Kastner et al., 1992).

### Example 2

### DEAR1 Expression

DEAR1 expression is limited to the ductal and glandular epithelium in normal tissues. DEAR1 is detected as a 4.4 kb primary transcript in multiple tissues on Northern analysis with other smaller transcripts expressed in either a developmental or tissue-specific pattern in skeletal muscle, placenta, brain and heart (FIG. 1C). Affinity purified anti-peptide antibodies were generated to the amino terminus of the DEAR1 protein. Peptide blocking experiments, performed in HMECs to confirm the specificity of the novel antibody, were indicative that the N-terminal DEAR1 antibody detects the predicted 54 kD full-length protein and that binding is specifically competed away in the presence of excess DEAR1 peptide (FIG. 1D). In addition, transient transfection assays using HA-tagged DEAR1 constructs introduced into 293T cells specifically detected the appropriate sized transcript (FIG. 1E). Western analysis confirmed that DEAR1 is expressed in all normal tissues analyzed (FIG. 1F). However, DEAR1 expression is localized to the ductal and glandular epithelium. Immunohistochemical analysis of a normal-tissue microarray (Biogenex) detected DEAR1 expression limited to the epithelial lining of the ducts and glands in the majority of normal tissues examined, including bladder, gall bladder, kidney, prostate, pancreas and salivary gland (FIG. 1G [i-vi, respectively]).

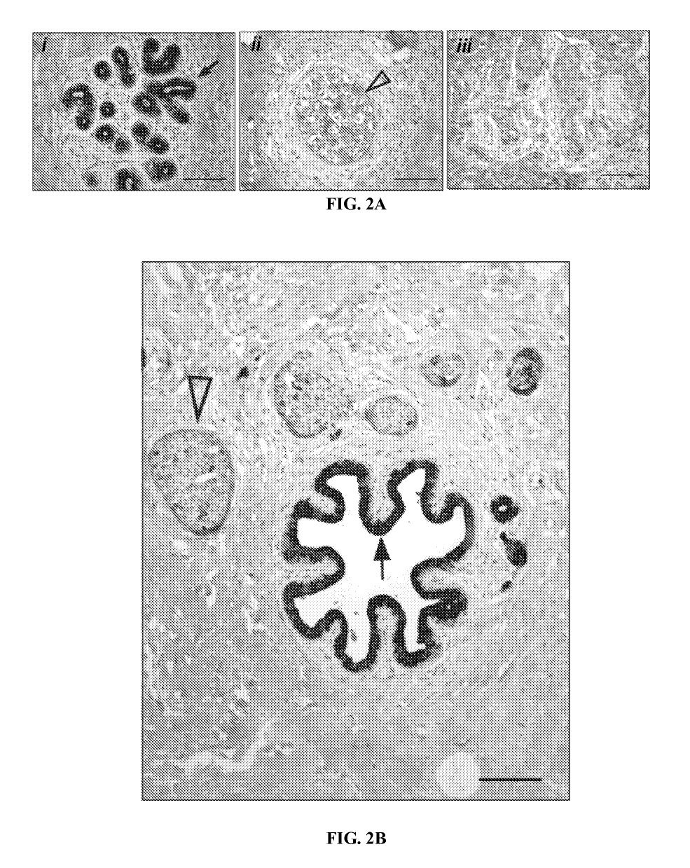

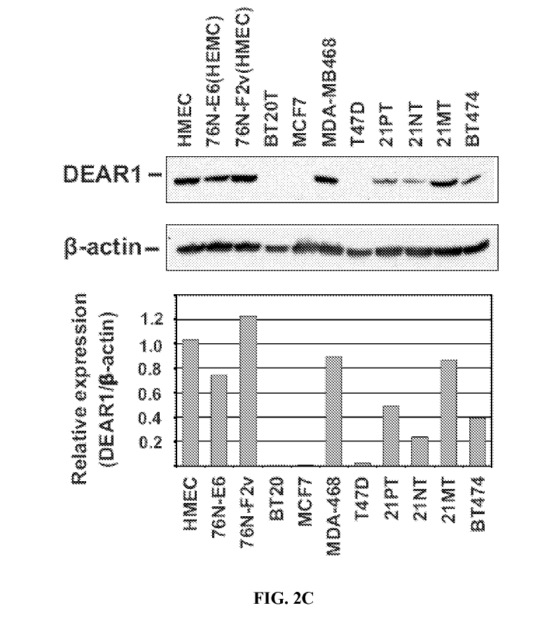

DEAR1 expression is downregulated in breast carcinoma cell lines and in transition to ductal carcinoma in situ. DEAR1 expression was examined by immunohistochemistry on a series of 14 ductal carcinoma in situ (DCIS) samples for which associated adjacent normal epithelium as well as the corresponding invasive cancer from the same individual. High levels of staining were observed in normal mammary ductal structures consistent with normal tissue microarray data (FIG. 2A[i], and FIG. 2B). However, 10/14 (71%) specimens showed loss or downregulation of DEAR1 expression in the transition from normal epithelium to DCIS (FIGS. 2A-2B). In high grade DCIS, DEAR1 expression was diminished at the basement membrane, with focal positivity in the center of the DCIS lesions (FIG. 2A[ii]). In specimens demonstrating downregulation of DEAR1 in DCIS transition, 5/10 specimens (50%, for which invasive carcinoma was available for analysis) showed loss or downregulation in the adjacent invasive carcinoma (FIG. 2A[iii]) with the remaining 5/10 invasive lesions positive for DEAR1 staining DEAR1 expression was also examined in normal HMECs, immortal HMEC variants as well as in breast carcinoma cell lines by Western blot analysis. Results indicated that DEAR1 expression was absent or downregulated in 6/8 (75%) breast carcinoma cell lines including two of three 21T series cell lines derived from a 36 year old with infiltrating ductal adenocarcinoma as compared with normal or immortalized HMECs (FIG. 2C) (Band et al., 1989; Band et al., 1990)

### Example 3

### DEAR1 is Mutated and Deleted in Breast Cancer

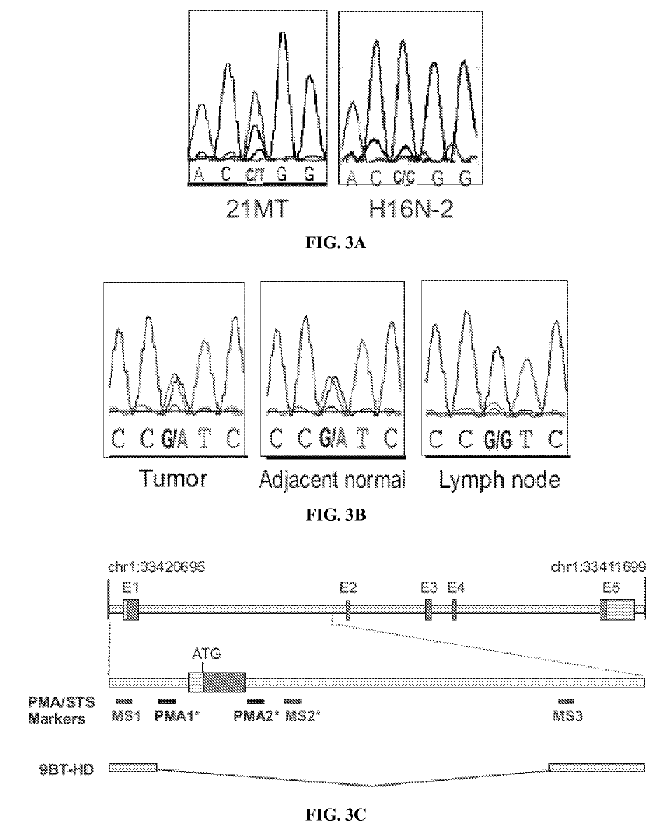

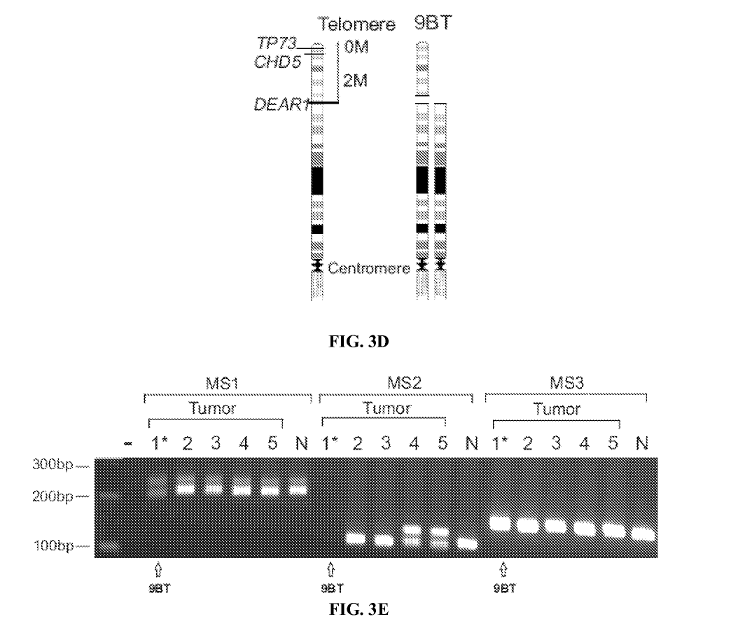

Mutational analysis was conducted on twelve breast cancer cell lines (itemized in Methods) as well as three cell lines of the 21T series (21NT, 21PT and 21MT) by DHLP and direct sequencing. Significantly, all of the cells lines in the 21T series contained identical nonconservative missense mutations in exon 3 within codon 187 (CGG→TGG, R187W) in the coiled-coil domain not observed in 136 normal alleles or the SNP database (FIG. 3A). The mammary epithelial cell strain (H16N-2) derived from normal breast epithelium of the same patient as the 21T series lines, did not contain the codon 187 mutation, indicative that the genetic alteration in the 21T series is not a rare polymorphism, but rather a tumor-derived mutational event (FIG. 3A, TABLE 1).

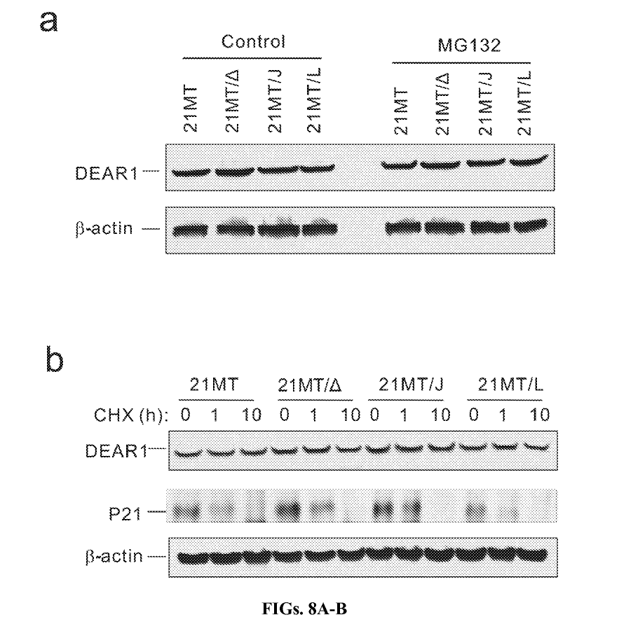

The R187W mutation falls between the two coils of the coiled-coil domain based on Parcoil (available on the world wide web at paircoillcs.mit.edu/cgi-bin/paircoil) and therefore might be predicted to affect protein binding to DEAR1. The mutation, however, does not affect protein stability following cycloheximide treatment. To investigate if the R187W mutation observed in the 21T series cell lines as well as a primary breast tumor (both from young women) affected DEAR1 protein stability, blocking proteasome-mediated degradation was first examined in stable transfectants and controls. Wild-type transfectants 21MT/J, and 21MT/L as well as mutant R187W transfectant 21MT/Δ and 21MT cells were treated with the proteasome inhibitor MG132. Results also indicated that there were no changes in protein expression levels following MG132 treatment (FIG. 8A). A cycloheximide chase experiment was then performed, in which 21MT, 21MT/J, 21MT/L and 21MT/187A were exposed to cycloheximide (50 μg/ml) for various times (0.5, 1, 1.5, 2, 4, 6, 8, and 10 h). Results indicate that DEAR1 expression in cells with the mutant allele did not show loss of stability over time in the presence of cycloheximide even after 10 h. FIG. 8B illustrates the 1 and 10 hr time points for all cell lines mentioned. Thus, these data indicate that the R187W mutation did not affect protein stability.

In addition to the 21T series mutations, breast cancer cell line MDA-MB-468 contained an intronic alteration not observed in the SNP database or in control lymphocytes (TABLE 1).

TABLE 1
DEAR1 Genetic Alterations in Breast Cell Lines

| | | | Absence of |
|---|---|---|---|
| | | | Alteration in |
| | | | Control |
| | | Presence of | Lymphocytes |
| Breast Tumor/ | | Alteration in | (Number of |
| Cell Line | Genetic Alteration | SNP Database | samples screened) |
| Normal Breast | None | N/A | N/A |
| Epithelium | | | |
| H16N-2 | | | |
| 21PT | Codon 187 mutation | No | 136 normal |
| | CGG→TGG, R→W | | alleles |
| 21NT | Codon 187 mutation | No | 136 normal |
| | CGG→TGG, R→W | | alleles |
| 21MT | Codon 187 mutation | No | 136 normal |
| | CGG→TGG, R→W | | alleles |
| MDAMB468 | G→A Intron Nt 28 | No | 114 normal |
| | ds exon 2 | | alleles |

Sequence analysis of 55 primary breast tumors obtained from the University of Texas M. D. Anderson Cancer tumor bank revealed that 13% contained genetic alterations in DEAR1 including three missense mutations, three intronic alterations and a silent mutation not observed in screening controls or the SNP database (TABLE 2). One missense mutation was observed in a breast tumor derived from a 36-year old female, occurring one nucleotide downstream of the 21MT mutation and thereby altering the same codon 187 (CGG→CAG, R187Q) as the 21MT cell line mutation. This mutation was observed in adjacent tissue but not in 136 normal alleles or the SNP database. Two missense mutations were identified in later onset breast tumor samples, both affecting exon 5 (GTC→ATC, V473I and GTC→ATC, V473I) (TABLE 2) and present in both tumor and adjacent normal samples but not in controls or the SNP database. In addition, the exon 5 mutation was not observed in normal lymph node from the same individual whose tumor contained the codon 473 mutation, indicative that the sequence alteration in the tumor was a somatic mutation of the DEAR1 sequence (FIG. 3B).

TABLE 2
DEAR1 Genetic Alterations in Breast Tumors

| | | | Absence of |
|---|---|---|---|
| | | | Alteration in |
| | | | Control |
| | | Presence of | Lymphocytes |
| Breast Tumor/ | | Alteration in | (Number of |
| Cell Line | Genetic Alteration | SNP Database | samples screened) |
| Breast Tumor | Codon 187 mutation | No | 136 normal |
| (S04T) | CGG→CAG, R→Q | | lymphocyte |
| | | | alleles |
| Breast Tumor | Codon 473 mutation | No | 80 normal |
| (B17T) | GTC→ATC, V→1 | | lymphocyte |
| | | | alleles; |
| | | | also not |
| | | | patients |
| | | | normal |
| | | | lymph node |
| Breast Tumor | Codon 350 mutation | No | 138 normal |
| (K06T) | GTC→G/ATC, | | lymphocyte |
| | V→V/I | | alleles |
| Breast Tumor | Codon 198 silent | No | ND |
| (S03T) | mutation | | |
| | GAG→GAA | | |
| Breast Tumor | Homozygous | No | N/A |
| (9BT) | deletion | | |
| Breast Tumor | G→A Intron Nt 28 | No | 114 normal |
| (S02T) | ds exon 2 | | lymphocyte |
| | | | alleles |
| Breast Tumor | G→A Intron Nt 28 | No | 114 normal |
| (S487T) | ds exon 2 | | lymphocyte |
| | | | alleles |
| Breast Tumor | G→A Intron Nt | No | 106 normal |
| (K09T) | 12 ds exon 1 | | lymphocyte |
| | | | alleles |

Significantly, a homozygous deletion (HD) was also identified in a primary tumor (9BT) obtained from a 39-year old with triple negative breast cancer. The deletion maps within the core promoter region of DEAR1 using Pyrosequencing Methylation Analysis (PMA) (assays 1 and 2, TABLE 3) on bisulfite-treated DNA (FIGS. 3C-3D; TABLE 3). Genomic PCR confirmed the homozygous deletion using STS markers that spanned microsatellite sequences MS1 and MS2, located upstream of the DEAR1 core promoter and in the first intron, respectively (FIGS. 3C-D). Results indicated that the MS1 region upstream of the 5′ UTR was retained in the 9BT sample (FIG. 3D), thus, mapping the breakpoint distal to MS1 and spanning the region identified by PMA. The distal boundary of the deletion was identified using primers which detected microsatellite sequence MS3 downstream of MS2 in intron 1, indicative that the HD encompassed the promoter and exon 1 with retention of exon 2 (MS3). Subsequent PMA detected a deletion of both the CHD5 and p73 genes which lie distal to DEAR1 in chromosome 1p, suggestive of a terminal deletion of one allele with a breakpoint within the DEAR1 promoter which then resulted in LOH encompassing two distal candidate tumor suppressors on chromosome 1p (FIG. 3E). Significantly, the HD was detected by two separate methodologies, indicating a breakpoint in both alleles within the DEAR1 promoter region. Thus, within a region of LOH for breast cancer and multiple epithelial tumors, a HD was identified in an early onset breast tumor. Additionally, because PMA detected heterozygous deletion of distal genes to DEAR1, and genomic PCR detected the HD limited to the DEAR1 promoter and exon 1, these results are consistent with a microdeletion in one allele and terminal deletion with breakpoint in the promoter of DEAR1 in the second allele, thereby deleting the entire DEAR1 coding region as well as the distal arm (FIG. 3D). The PMA analysis of 14 breast cancer cell lines and 20 tumor samples did not reveal promoter methylation in any of the samples.

TABLE 3
Primers used to identify a homozygous deletion (HD) in breast tumors

| | | | | SEQ ID | |
|---|---|---|---|---|---|
| | Start-End* | Primer | Sequence SEQ ID NO: | NO: | Amplicon |
| MS1 | chr1:33420695- | Forward | 5′-TCCCTTATCCCCTCTCCATC-3′ | 15 | 215 bp |
| | 33420909 | | | | |
| | | Reverse | 5′-TTAAGGAGTGCTTGGGGAGA-3′ | 16 | |
| MS2 | chr1:33418095- | Forward | 5′-GCTCAAATATCCTCTCCGTGA-3′ | 17 | 150 bp |
| | 33418244 | | | | |
| | | Reverse | 5′-CCAAGGGGGTGGGTATAAAA-3′ | 18 | |
| MS5 | chr1:33411516- | Forward | 5′-GAAAAGCAAGGCTTGACCAG-3′ | 19 | 184 bp |
| | 33411699 | | | | |
| | | Reverse | 5′-TGCCTGCTCACATTTGTTTC-3′ | 20 | |
| PMA1 | Chr1:33420150- | Forward | 5′-BIO-TTTYGGGTTGAGAGGTTG-3′ | 21 | 86 bp |
| | 33420235 | | | | |
| | | Reverse | 5′ -ACCCCAAACCCTAACCCA-3′ | 22 | |
| | | Sequencing | 5′-CCAAACCCTAACCCAAC-3′ | 23 | |
| PMA4 | chr1:33419182- | Forward | 5′-TGTAYGAGTAGTATTAGGTTA-3′ | 24 | 106 bp |
| | 33419265 | | | | |
| | | Reverse | 5′-GACGGGACACCGCTGATCGTTT | 25 | |
| | | | ACCCCTTCCCCAAACAAACC-3′ | | |
| | | Sequencing | 5′-GAGTAGTATTAGGTTAT-3′ | 26 | |

*Note:
based on the March 2006 human reference sequence: NCBI Build 36.1

### Example 4

### DEAR1 Restores Acinar Morphogenesis in 3-D Basement Membrane Culture

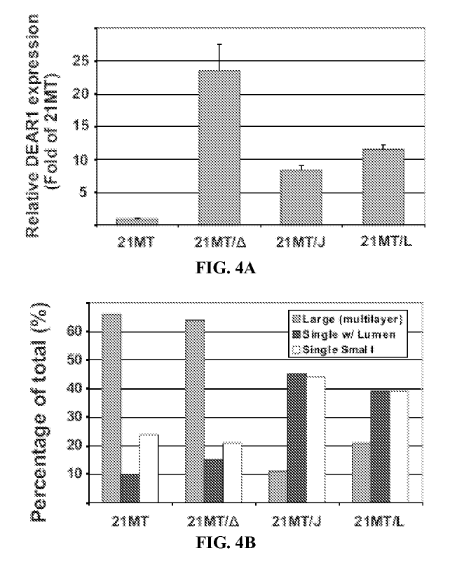

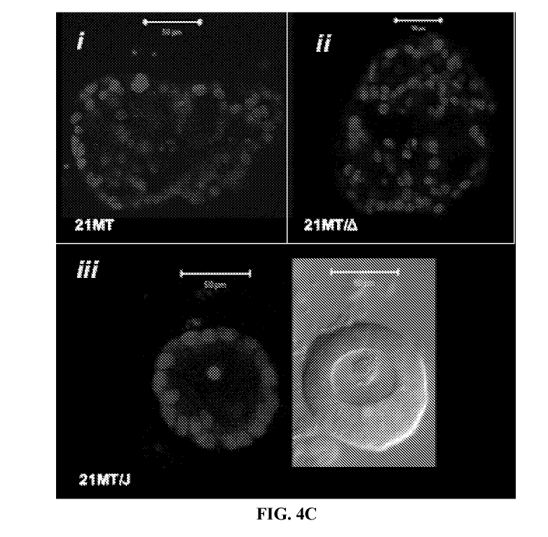

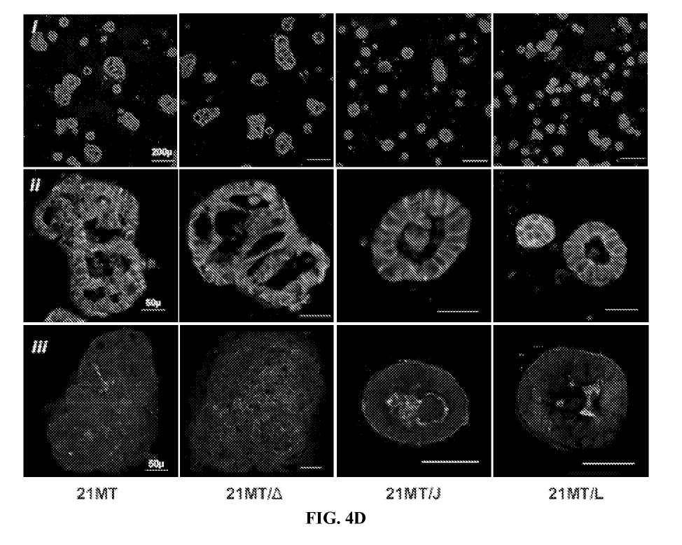

In order to determine if the mutations in DEAR1 were important to the genesis or progression of breast cancer and not mere “passenger” mutations, functional assays were performed. To determine the effect of genetic complementation of the missense mutation affecting codon 187 in the breast cancer progression model as well as in a breast tumor sample, full length DEAR1 wild-type and R187W mutant cDNA were introduced into 21MT to generate stable transfectants. Quantitative RT-PCR confirmed upregulation of DEAR1 RNA levels following stable transfection (FIG. 4A). cDNA sequencing confirmed expression of predominant wild-type DEAR1 transcripts in 21MT/J and 21MT/L transfectants and as well as the R187W mutant transcripts in control 21MT/Δ. Protein expression levels on Western analyses were very similar among transfectants and controls, including HMECs (FIG. 2C, FIGS. 8A-B). 21MT cells, wild-type transfectants (21MT/J) and (21MT/L) as well as R187W transfectant 21MT/Δ were then plated in 3-D basement membrane culture. Results indicated that >60% of 21MT cells in 3-D culture formed large, disorganized structures as determined by staining with propidium iodide followed by visualization using confocal microscopy (FIG. 4B). Significantly, introduction of the tumor-associated R187W missense mutation in 21MT/Δ also resulted in a similar percentage of large, irregularly shaped multiacinar structures as observed in 21MT cells (FIG. 4B). However, introduction of wild-type DEAR1 into 21MT cells resulted in acinar morphogenesis with >80% of wild-type transfectants producing small, spherical acini. Forty percent of these structures contained a central lumen surrounded by a single layer of polarized epithelial cells (FIG. 4B, 4D[i]) unlike the vast majority of multiacinar structures observed in 21MT and missense mutant controls as visualized by confocal and differential interference contrast (DIC) microscopy (FIG. 4B, 4C, 4D[i]). Thus, the morphological appearance of wild-type transfectants was strikingly similar to normal acini formed by HMECs in 3-D culture.

On day 9 of 3-D culture, DEAR1 transfectants (n=50) had a median diameter in 3-D culture of 71.0 μm (interquartile range, 58.6 to 91.9 μm; range, 43.2 to 167.3 μm) for full-length DEAR1 transfectant (J) and 69.8 μm (interquartile range, 60.2 to 85.0 μm; range, 40.7 to 139.8 μm) for transfectant (L). The diameter of 21MT structures in 3-D culture measured 108.6 μm (interquartile range, 81.3 to 166.6 μm; range, 48.8 to 394.1 μm; n=50) which was significantly different from acini formed by transfectants with DEAR1 (using a Mann-Whitney statistical analysis, p<0.0001). Similarly, transfectant 21MT/Δ containing the codon 187 missense mutation resulted in structures (median, 128.5 μm; interquartile range, 88.9 to 176.0 μm; range, 38.1 to 304.6 μm; n=50) which by size and morphology closely resembled 21MT cells in basement membrane culture and which were significantly different from DEAR1 wild-type transfectants (p<0.0001). Staining with E cadherin allowed the visualization of cell-cell contacts and emphasized the distorted cell structures in 21MT and 21MT/Δ in which cells of various sizes and shapes were observed with many misshapen cells visualized by confocal microscopy (FIG. 4D[ii]). 21MT and 21MT/Δ transfectants in 3-D culture also showed diminished polarized expression of α-6-integrin, which is normally expressed on the basolateral surface at the cell membrane (FIG. 4D). In contrast to 21MT and 21MT/Δ, E-cadherin staining in wild-type DEAR1 transfectants was localized at cell-cell contacts in acini which displayed uniform cell size and clear basal orientation of nuclei with increased basal localization of α-6-integrin, indicative of a restoration of ordered acinar architecture (FIG. 4D[iii]). Furthermore, Caspase 3 staining detected active luminal apoptosis in day 13 acinar structures in wild-type transfectant clones, recapitulating a defined event in normal mammary acinar morphogenesis (FIG. 4D[iii]). In addition, results indicated no discernible difference in Ki67 staining in 3-D cultures of 21MT versus wild-type or mutant transfectants at day 13 when wild-type transfectants were undergoing active luminal apoptosis (FIG. 9), suggesting that DEAR1's influence on apoptotic rather than proliferative pathways is more evident in this model system. Thus, the introduction of wild-type DEAR1, resulted in restoration of normal epithelial acinar architecture, a reinitiation of apicobasal polarity as well as a clearing of luminal space, providing evidence for the role of DEAR1 in the dominant regulation of acinar morphogenesis and indicative that the 21MT missense mutant phenotype could be rescued by the introduction of wild-type DEAR1.

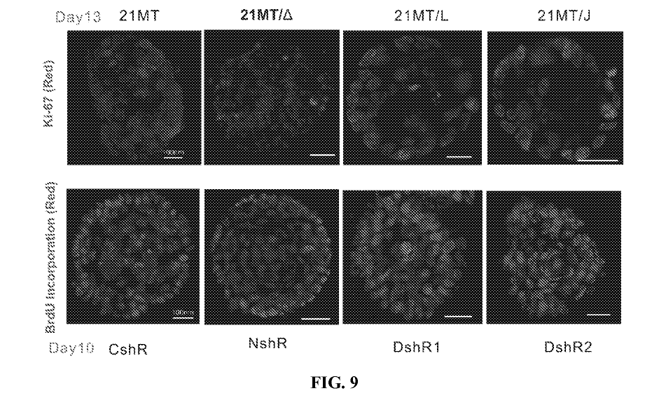

Similar results were obtained by transient transfection of DEAR1 into MCF-7 cells, which have very low to undetectable DEAR1 expression (FIG. 2C), in which transient expression of DEAR1 could partially restore acinar morphogenesis in this cell line (FIGS. 10A-10B). Western analysis confirmed DEAR1 upregulated expression in MCF-7 cells post transfection of pCMV-DEAR1 (FIG. 10A). Results from growth in 3D culture indicate that MCF-7 cells grow similarly to 21MT in that by day 19, they have a very irregular growth pattern with lack of normal acinar structures (FIG. 10B). However, introduction of DEAR1 resulted in an increase in more uniform and polar acinar structures, some of which had discrete lumen (FIG. 10B). Thus, even in transient assay we were able to document that DEAR1 could partially revert aberrant MCF-7 growth in 3D culture.

### Example 5

### Knockdown of DEAR1 in Human Mammary Epithelial Cells Recapitulates the Phenotype of 21MT in 3-D Culture

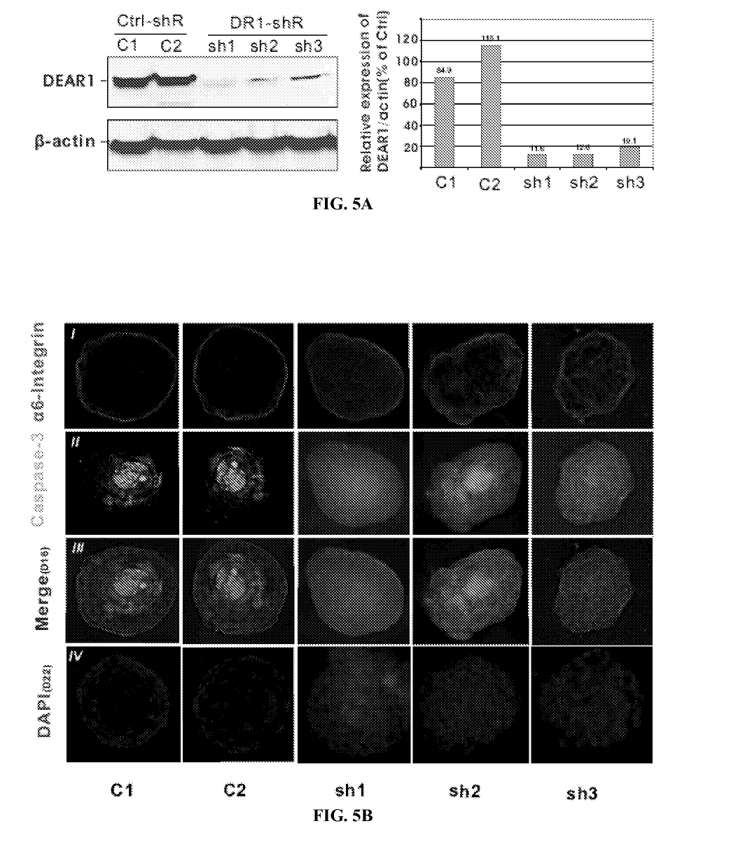

To determine the effect of loss of function of DEAR1 in normal mammary differentiation, DEAR1 expression was silenced in immortalized human mammary epithelial cells (76N-E6 cells) using lentiviral short hairpin RNA (shRNA). Three shDEAR1 clones as well as control shRNA clones were examined by western analysis (FIG. 5A) and for growth in three dimensional culture (FIG. 5B). Results indicated that DEAR1 stable knockdown clones (3/3), which were extensively silenced for DEAR1 expression (FIG. 5A), failed to form normal acini in 3-D culture with irregular, asymmetric structures visible following 12 days in 3-D culture (FIG. 5B). Furthermore, cells within asymmetric structures appeared disorganized with ubiquitous staining for alpha-6-integrin, indicative of loss of polarity. Diffuse low to moderate staining for Caspase 3 was also observed in shDEAR1 clones at day 16 during which time control HMECs demonstrated active luminal apoptosis. These results indicate that without DEAR1, apoptosis is not restricted to the lumen. shDEAR1 clones failed to form lumen even after 22 days in culture as compared with control knockdown clones which formed discrete lumen by the same time point (FIG. 5B[iv]). In addition, Ki67 staining and BrdU incorporation in day 10 acinar structures indicated no significant difference in proliferation between knockdown and control clones (FIG. 9). Thus, stable silencing of DEAR1 in immortalized, nontransformed human mammary epithelial cells disrupted normal acinar morphogenesis and recapitulated the phenotype observed in 21MT.

### Example 6

### Loss of DEAR1 Expression in Early Onset Breast Cancers Correlates with the Triple Negative Phenotype of Breast Cancers with Poor Prognosis and Strong Family History of Breast Cancer

Because both DEAR1 mutations and a homozygous deletion were observed in primary tumors from young women, and because the functional significance of complementation of a tumor-derived mutation and in vitro silencing of the gene was demonstrated, these data indicate that DEAR1 is involved in the underlying genetic etiology of early onset breast cancer. To address the clinical significance of DEAR1 in early onset breast cancer, a well characterized tissue array from a cohort of 158 premenopausal women with onset of breast cancer between the ages of 25-49 years was screened by immunohistochemistry for DEAR1 expression (Parikh et al., 2008). All of the tissue array samples were from stage I or II breast cancers treated with breast conservation surgery and postsurgical radiation therapy (TABLE 4). Significantly, all progressed to invasive disease even though 72% of samples were from node negative breast cancers. Interrogation of this array using the N-terminal DEAR1 antibody developed, identified 56% of the tumor samples with complete loss of DEAR1 expression, while 44% retained expression.

TABLE 4
Patient and tumor characteristics stratified by DEAR1 expression

| | | DEAR1 Expression | | |
|---|---|---|---|---|
| Features | Number | Negative | Positive | p |
| Histology | | | | 0.6582 |
| Ductal | 100 | 55(83%) | 45(88%) | |
| Lobular | 5 | 2(3%) | 3(6%) | |
| Others | 12 | 9(14%) | 3(6%) | |
| Tumor Size | | | | 0.1463 |
| T₁ | 75 | 47(75%) | 28(61%) | |
| T₂ | 34 | 16(25%) | 18(39%) | |
| Nodal Status | | | | 1.0000 |
| Negative | 74 | 43(73%) | 31(72%) | |
| Positive | 28 | 16(27%) | 12(28%) | |
| ER | | | | 0.4253 |
| Negative | 71 | 41(68%) | 30(60%) | |
| Positive | 39 | 19(32%) | 20(40%) | |
| PR | | | | 0.0321 |
| Negative | 68 | 43(70%) | 25(49%) | |
| Positive | 44 | 18(30%) | 26(51%) | |
| HER2 | | | | 0.7526 |
| Negative | 103 | 57(92%) | 46(90%) | |
| Positive | 10 | 5(8%) | 5(10%) | |
| Triple negative | | | | 0.0362 |
| No | 58 | 26(43%) | 32(64%) | |
| Yes | 52 | 34(57%) | 18(36%) | |
| p53 | | | | 1.0000 |
| Negative | 85 | 46(75%) | 39(76%) | |
| Positive | 27 | 15(25%) | 12(24%) | |
| Strong family history | | | | 0.0139 |
| No | 92 | 46(73%) | 46(92%) | |
| Yes | 21 | 17(27%) | 4(8%) | |
| BRCA1 mutation | | | | 0.6347 |
| No | 47 | 28(88%) | 19(95%) | |
| Yes | 5 | 4(12%) | 1(5%) | |
| BRCA2 mutation | | | | 0.5173 |
| No | 50 | 30(94%) | 20(100%) | |
| Yes | 2 | 2(6%) | 0(0%) | |

Clinical parameters for the cohort under study were analyzed for statistical significance with DEAR1 expression. The analysis included two groups, those samples scored as either focal or diffuse positive in the positive group and all samples scored as total absence of staining in the negative group. Thirty-five of the 158 total samples were not scorable due to loss of tissue. Results on 123 samples indicated that DEAR1 loss of expression did not correlate significantly with tumor size (correlation coefficient: r=0.15), lymph node metastasis (r=0.01), race (r=−0.03), ER (r=0.09), HER2 (r=0.03), or p53 (r=−0.01) expression status (TABLE 4). DEAR1 loss of expression did not correlate with BRCA1 or BRCA2 mutation, but rather, loss of expression correlated with a strong family history of breast cancer in this young cohort (r=−0.24, p=0.0139). Seventeen of 21 individuals represented on the tissue array with a strong history of breast cancer in their families were negative for DEAR1 staining. Furthermore, loss of DEAR1 expression correlated significantly with loss of progesterone receptor expression and with the triple negative phenotype (ER⁻, PR⁻, HER2⁻) of breast cancers (r=0.21, p=0.0362), a subgroup common in BRCA1 mutation carriers and identified by gene expression profiling as breast cancers of poor prognosis and for which few treatment options exist (TABLE 4) (Sorlie et al., 2001). Together, the loss of expression of DEAR1 in the majority of early onset cases examined and its correlation with family history and the triple negative phenotype strongly supported that this gene as a candidate biomarker in early onset breast tumors.

### Example 7

### DEAR1 is an Independent Predictor of Local Recurrence-Free Survival in Early Onset Breast Cancer

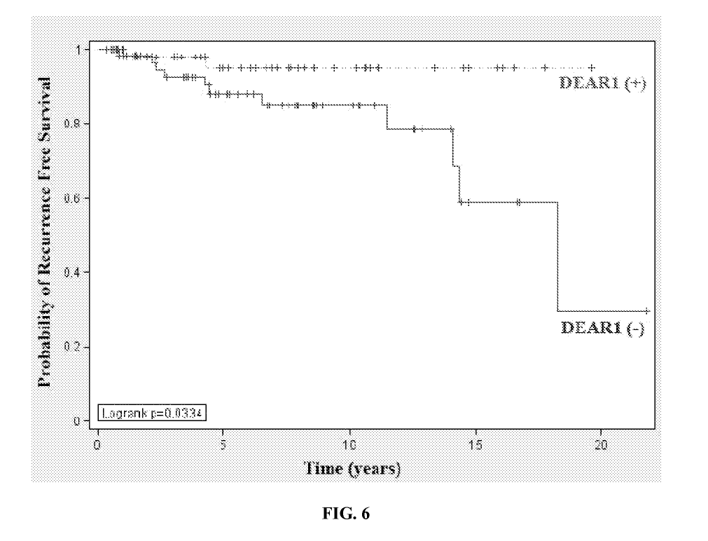

Although loss of DEAR1 expression did not correlate with distant metastasis or survival in this young cohort of women with early stage breast cancer, loss of DEAR1 expression on immunohistochemical staining significantly predicted local recurrence. At 5 years follow-up, DEAR1 positive expression correlated with a 95% local recurrence-free survival; and significantly, this survival rate did not change in the cohort used herein for over 15 years postsurgical follow-up. For those samples demonstrating loss of expression of DEAR1, recurrence-free survival fell to 80% at 10 years and 58% at 15 years (p=0.034) (FIG. 6). Thus, these data indicate that DEAR1 is an independent predictor of local recurrence in early onset breast cancers and suggest that DEAR1 negative staining on immunohistochemistry could be a significant marker to stratify early onset breast cancer patients for increased vigilance in follow-up and adjuvant therapy.

### Example 8

### Methods

Cell Lines and Tumor Samples. The 21NT, 21PT and 21MT lines were propagated in Dulbecco's modified eagle's medium/F12 (DMDM/F12) supplemented with 10% fetal bovine serum, 1 μg/ml insulin, 12.5 μg/ml EGF and 1 μg/ml hydrocortisone. Human mammary epithelial cells (HMECs) (ATCC) and the immortalized breast epithelial line MCF-10A were propagated in EMGM medium according to ATCC protocols. The remainder of breast carcinoma cell lines (T47D, BT474, MCF-7, H38, Zr75T, MDA-MB-157, HBL100, HS578T, BT20T, MDA-MB-231, and MDA-MB-436) used for mutations screens and expression studies were grown in DMEM/F12 supplemented with 10% fetal bovine serum.

PCR Select Subtractive Hybridization. Total RNA was isolated with TRIzol (Invitrogen, Carlsbad, Calif.) with subsequent isolation of the poly-A⁺ population using oligo dT cellulose. The PCR-Select suppression subtractive hybridization assay (Clontech Laboratories, Palo Alto, Calif.) was used to identify cDNAs differentially expressed between the microcell hybrid lines SN19(3)EEE [driver] and SN19(3i)YY [tester] (Sanchez et al., 1994; Lott et al., 1998; Lovell et al., 1999; Zhang et al., 2007). PCR products from the secondary PCR reactions were cloned into the pCRTMII vector (Invitrogen, Carlsbad, Calif.).

Library Screening. To identify a full-length cDNA clone of DEAR1, a human retinoic acid induced neuroepithelial cell library cloned into the ZAP Express XR vector (Stratagene, La Jolla, Calif.) was screened with a ³²P end-labelled oligonucleotide corresponding to the 5′ end of the partial cDNA (DEAR1 FOR-5′ TTGATCCAAGGATGTGACATG 3′ (SEQ ID NO:1)). Positive plaques were excised and confirmed by PCR using the DEAR1-FOR and DEAR1-REV (5′ GTGACCACTGTGGACTGGG 3′ (SEQ ID NO:2)). The ExAssist helper phage was used to excise the pBK-CMV expression vector positive ZAP Express clones according to the manufacturer's protocol. Sequencing of this phagemid identified an alternative splice form of DEAR1 (exons 1-3 and 5). Screening of the RPCI 4 PAC library (available at bacpac.chori.org) using the phagemid insert end-labeled with ³²P was performed to identify a genomic clone of DEAR1.

Generation of a Full-Length Expression Construct. To obtain a cDNA with all exons, one through five, the IMAGE clone 3355572 was obtained from ATCC (Manassas, Va.). Using this clone as a template, the open reading frame was amplified by PCR (forward primer-M13 5′ GTAAAACGACGGCCAGT 3′ (SEQ ID NO:3) and reverse primer-7b5-2032-AS 5′ GTCTTAGGCCATGGGACATAAGAG 3′ (SEQ ID NO:4)). This yielded a 1972 bp product that was subsequently ligated into pBK-CMV digested with EcoRI/XhoI.

FISH Mapping. FISH mapping of PAC clones was performed according to previously published protocols (Sanchez et al., 1994).

Promoter Methylation and Deletion Analysis. Pyrosequencing-based Methylation Analysis (PyroMethA) was performed according to the method of Colella et al. (2003). Primers for deletion studies as well as promoter analysis by PyroMethA are available upon request.

Mutation Screening. For exons two through five, 100 ng of genomic DNA was amplified in a using AmpliTaq Gold Taq polymerase (Applied Biosystems, Foster City, Calif., USA). Since the amplification of Exon 1 proved difficult and inconsistent under standard conditions due in part to its G-C content, alternative conditions were used (Kogan et al., 1987). The intronic primer sequences are as follows: Exon 1-forward: GCTCCTACCCCTGCCTGT (SEQ ID NO:5), reverse: CCCCACCTCCAGCCC (SEQ ID NO:6); Exon 2-forward: GCAGTGGTCAGGGCTGAATG (SEQ ID NO:7), reverse: CCTTCTTCCCCAGCTGGC (SEQ ID NO:8); Exon 3-forward: CTGTGGTGTCAAGGCTCTCGA (SEQ ID NO:9), reverse: CTCTGCTAAGGATCCCATCTG (SEQ ID NO:10); Exon 4-forward: CACATCCTATGCCAGCTGC (SEQ ID NO:11), reverse: CAAGGCACTCAGCACATTC (SEQ ID NO:12); Exon 5-forward: CTGGAAGGACCTTAACCACCA (SEQ ID NO:13), reverse: CTATCTTCCGGGCAGGGCTC (SEQ ID NO:14). The expected product sizes are: 585 bp for exon 1, 250 bp for exon 2, 420 bp for exon 3, 329 bp for exon 4 and 800 bp for exon 5. PCR products were treated with ExoSAP (USB, Cleveland, Ohio) and submitted to the M.D. Anderson DNA core facility for sequencing or denaturing high-performance liquid chromatography (DHPLC). Electropherograms were analyzed using either Sequencher or Lasergene software packages.

Antibody Production. Lasergene sequence analysis software was utilized to identify non-conserved regions of DEAR1 that also scored highly for antigenicity. Peptide synthesis and polyclonal antibody production was performed by Bethyl Laboratories. Rabbits were immunized with the DEAR1 peptide conjugated to keyhole limpet hemocyanin (KLH). DEAR1 antibodies were affinity-purified.

Transient transfection, Whole cell extracts and Western blotting. For detection of exogenous HA-DEAR1, 293T cells were seeded in a 24-well plate at 4×10⁴ cells/well over night before transfection. 0.2 μg of pCMV-HA/DEAR1 plasmid and FuGene6 transfection reagent (1 μg:3 μl) (Roche Applied Science) were added in each well. After 24 h culture, the cells were scraped into 60 μl of 1×SDS sample buffer. For whole cell lysates, cell lines were grown exponentially, harvested and lysed in 1×SDS sample buffer. Equal amounts of protein per lane were loaded on 4-20% SDS-PAGE gradual gels (Pierce), transferred to membranes, and analysed using antibodies against DEAR1 and β-actin (Sigma). For peptide-blocking experiment, DEAR1 antibody was mixed with 5× peptide of DEAR1 (v/v) for 2 hrs in room temperature, prior to incubation with membrane.

Stable Transfection. Transfection of the pBK-CMV/Δ187DEAR1 and the pBK-CMV/DEAR1 constructs into 21MT using Lipofectamine 2000 (Invitrogen, Carlsbad, Calif.). Stable transfectants were isolated as single colonies following selection in G418 (500 μg/ml).

DEAR1 Stable Knockdown. MISSION® shRNA lentiviral vectors expressing nontarget control shRNA or DEAR1 shRNAs and packaging vectors were purchased from Sigma (NM_{—}018207). Cotransfection of retroviral and packaging vectors into HEK293T packaging cells for production and packaging of retroviruses was performed according to the manufacturer's recommendations. The supernatant containing virus was harvested and filtered 48 h-72 h after transfection. Viral supernatant was infected into 76N-E6 cells in the presence of 8 ug/ml hexadimethrine bromide. Stable clones were selected using puromycin (2 μg/ml).

Three Dimensional Culture. Three dimensional culture assays for acini formation were performed as described by Debnath et al. (2002).

Immunohistochemistry. Immunohistochemistry was performed on 5 μm sections cut from formalin fixed, paraffin-embedded tissue. Following deparaffinization and rehydration, sections were subjected to antigen retrieval in either 10 mM sodium citrate, pH 6.0, for 15 minutes in a microwave pressure cooker for the DEAR1 antibody or Protease XXIV (BioGenex, San Ramon, Calif.) for 10 minutes at room temperature for the α-laminin-5 antibody (Chemicon International, Temecula, Calif.). Subsequent staining procedures were performed according to the Super Sensitive Non-Biotin HRP/DAB Detection System (BioGenex, San Ramon, Calif.) with the primary antibodies diluted 1:200 in Common Antibody Diluent (BioGenex, San Ramon, Calif.). Sections were counterstained with Mayer's hematoxylin and mounted with Permount (Fisher Scientific, Hampton, N.H.). Human tissue was obtained with appropriate IRB approval. DEAR1 expression was scored as negative expression when there was no detectable staining and positive expression when the staining was diffuse positive, focal positive or strong positive.

Statistical Analysis. A database containing DEAR1 status and relevant co-variables was assembled and analyzed using SAS Version 9.1 (SAS Institute, Cary, N.C.). All tests of statistical significance were two-sided. p-values less than 0.05 were considered statistically significant. Bivariate analyses for the association between co-variables and DEAR1 status included the chi-square and Fisher's exact test. Bivariate analyses for the associations between predictor variables and local and distant recurrence, and overall survival were conducted using the Kaplan Meier log-rank test and the chi-square test for linear trend. In the multivariate analysis, DEAR1 proportional hazards regression determined significant predictors of disease-free survival and overall survival at a p=0.05 level in the final model.

### VII. REFERENCES

The following references, to the extent that they provide exemplary procedural or other details supplementary to those set forth herein, are specifically incorporated herein by reference.
U.S. Pat. No. 4,196,265
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 6,358,683
U.S. Pat. No. 6,943,245
U.S. Patent Publn. 20030124543
U.S. Patent Publn. 2005/0171045
Band et al., Cancer Res., 50:7351-7357, 1990.
Band et al., Genes Chromosomes Cancer, 1:48-58, 1989.
Barany and Merrifield, Anal. Biochem., 95(1):160-70, 1979.
Bellus, J. Macromol. Sci. Pure Appl. Chem., A31(1): 1355-1376, 1994.
Bonnier et al., Int. J. Cancer, 62:138-144, 1995.
Borden, Biochem. Cell Biol., 76:351-358, 1998.
Borg et al., Genes Chromosomes Cancer, 5:311-320, 1992.
Campbell et al., Am. Rev. Respir. Dis., 130(3):417-423, 1984.
Cancer: Principles and Practice of Oncology, 7^{th} Ed., 2004.
Capaldi et al., Biochem. Biophys. Res. Comm., 76:425, 1977.
Clinical Oncology, 3^{rd} Ed., 2004.
Colella et al., Biotechniques, 35:146-150, 2003.
Darnell, In: Molec. Cell Biol., 3^{rd} Ed., W. H. Freeman, NY, 1990.
de la Rochefordiere et al., Lancet, 341:1039-1043, 1993.
Debnath et al., Cell, 111:29-40, 2002.
Debnath et al., Methods, 30(3):256-68, 2003.
European Appln. 320 308
European Appln. 329 822
Fidler et al., Adv. Cancer Res., 28:149-250, 1978.
Fish and Molitoris, N. Engl. J. Med., 330:1580-1588, 1994.
Fisher et al., Lancet, 338:327-331, 1991.
Foder et al., Science, 251:767-773, 1991.
Fowble et al., Int. J. Radiat. Oncol. Biol. Phys., 30:23-33, 1994.
Freemont, In: The RING finger. A novel protein sequence motif related to the zinc finger, Ann. NY Acad. Sci., 684:174-192, 1993.
Freifelder, Physical Biochemistry, 2^{nd} Ed., pages 238-246, 1982.
Frohman, In: Pcr Protocols: A Guide To Methods And Applications, Academic Press, N.Y., 1990.
Furuta et al., Proc. Natl. Acad. Sci. USA, 102:9176-9181.
GB Appln. No. 2 202 328
Gefter et al., Somatic Cell Genet. 3:231-236, 1977.
Goding, In: Monoclonal Antibodies: Principles and Practice, 2d ed., Orlando, Fla., Academic Press, pp. 60-61, 65-66, 71-74, 1986.
Gohji et al., Cancer Research, 56:3196, 1996
Gohji et al., J. Clin. Oncol., 18:2963, 2000
Gudjonsson et al., Methods, 30:247-255, 2003.
Hacia et al., Nature Genet., 14:441-449, 1996.
Haemmerlin et al., Int. J. Cancer, 27, 603-610, 1981.
Haffty et al., J. Clin. Oncol., 9:997-1003, 1991.
Haffty et al., Lancet, 359(9316)1471-7, 2002.
Harlow and Lane, In: Antibodies: A Laboratory Manual, Cold Spring Harbor Laboratory, 1988.
Harrold et al., Cancer J. Sci. Am., 4:302-307, 1998.
Hosaka et al., Gann, 69:273-276, 1978.
Innis, et al., In: PCR Protocols. A guide to Methods and Application, Academic Press, Inc. San Diego, 1990.
Isomura et al., Nucleic Acids Res., 20:5305-5310, 1992.
Izes et al., J Urol., 165(6 Pt 1):1908-13, 2001
Jemal et al., Cancer statistics CA: a cancer journal for clinicians, 53(1):5-26, 2003.
Kastner et al., Embo J., 11:629-642, 1992.
Killary et al., Proc. Natl. Acad. Sci. USA, 89: 10877-10881, 1992.
Kirshner et al., Proc. Natl. Acad. Sci. USA, 100:521-526, 2003.
Kogan et al., N. Engl. J. Med., 317:985-990, 1987.
Kohler and Milstein, Eur. J. Immunol., 6:511-519, 1976.
Kohler and Milstein, Nature, 256:495-497, 1975.
Kwoh et al., Proc. Natl. Acad. Sci. USA, 86(4):1173-1177, 1989.
Lagios, Surg. Clin. North Am., 70:853, 1990.
Le Douarin et al., Embo J., 14:2020-2033, 1995.
Liotta et al., Cancer Treatment Res., 40:223-238, 1988.
Lott et al., Cancer Res., 58:3533-3537, 1998.
Lovell et al., Cancer Res., 59:2182-2189, 1999.
Mark et al., Genet. Med., 1(3):98-103, 1999.
Maulard-Durdux et al., J. Clin. Oncol., 15:3446, 1997.
Merrifield, Science, 232(4748):341-7, 1986.
Milikan et al., Genes, chromosomes, & cancer, 25(4):354-61, 1999.
Muthuswamy et al., Nat. Cell Biol., 3:785-792, 2001.
Nicolson, Biochim. Biophy. Acta, 948:175-224, 1988.
Ohara et al., Proc. Natl. Acad. Sci. USA, 86:5673-5677, 1989.
Parikh et al., Int. J. Radiat. Oncol. Biol. Phys., 70:35-42, 2008.
PCT Appln. PCT/US87/00880
PCT Appln. PCT/US89/01025
PCT Appln. WO 88/10315
PCT Appln. WO 89/06700
PCT Appln. WO 90/07641
PCT Appln. WO 98/02539
Pease et al., Proc. Natl. Acad. Sci. USA, 91:5022-5026, 1994.
Petersen et al., Proc. Natl. Acad. Sci. USA, 89:9064-9068, 1992.
Pignon et al., Hum. Mutat.; 3(2):126-32, 1994.
Ragnarsson et al., Br. J. Cancer, 79:1468-1474, 1999.
Reddy et al., Dev. Biol., 148:107-116, 1991.
Reddy et al., Trends Biochem. Sci., 17:344-345, 1992.
Reichmann, Semin. Cancer Biol., 5:157-165, 1994.
Reymond et al., Embo J., 20:2140-2151, 2001.
Sambrook et al., In: Molecular Cloning: A Laboratory Manual, Vol. 1, Cold Spring Harbor Laboratory Press, Cold Spring Harbor, N.Y., Ch. 7,7.19-17.29, 1989.
Sanchez et al., Proc. Natl. Acad. Sci. USA, 91:3383-3387, 1994.
Shoemaker et al., Nature Genetics, 14:450-456, 1996.
Sorlie et al., Proc. Natl. Acad. Sci. USA, 98:10869-10874, 2001.
Stewart and Young, In: Solid Phase Peptide Synthesis, 2^{nd} Ed., Pierce Chemical Co., 1984.
Tam et al., J. Am. Chem. Soc., 105:6442, 1983.
Urano et al., Nature, 417:871-875, 2002.
Veronesi et al., J. Natl. Cancer Inst., 87:19-27, 1995.
Walker et al., Nucleic Acids Res., 20(7):1691-1696, 1992.
Wang et al., Proc. Natl. Acad. Sci. USA, 95:14821-14826, 1998.
Weaver et al., Cancer Cell, 2:205-216, 2002.
Weaver et al., J. Cell Biol., 137:231-245, 1997.
Wu and Wallace, Genomics, 4:560-569, 1989.
Zetter, N. Eng. J. Med., 322:605-612, 1990.
Zhang et al., Biochem. Biophys. Res. Commun., 360:531-538, 2007.
Zhou and Recht, Cancer, 101: 1264-1274, 2004.

1. An in vitro method of evaluating prognosis of a subject having cancer comprising determining if a cancer cell of the subject has a reduced level of DEAR1 expression and/or function as compared to a reference level, wherein a reduced level of DEAR1 expression and/or function indicates a poor prognosis.

2. The method of claim 1, wherein the method further comprises developing a treatment plan.

3. The method of claim 1, wherein the method is further defined as treating a subject having cancer and determined to have a reduced level of DEAR1 expression and/or function as compared to a reference level with a targeted therapy.

4. The method of claim 1, wherein determining if the cancer cell has a reduced level of DEAR1 expression and/or function comprises determining the level of DEAR1 expression.

5. The method of claim 1, wherein the poor prognosis comprises a high risk of local cancer recurrence.

6. The method of claim 1, wherein lack of detectable expression of DEAR1 indicates a poor prognosis.

7. The method of claim 1, wherein DEAR1 mRNA expression is evaluated.

8. The method of claim 1, wherein DEAR1 protein expression is evaluated.

9. The method of claim 7, wherein DEAR1 mRNA expression is evaluated using Northern blotting, RT-PCR, quantitative RT-PCR, nuclease protection, an in situ hybridization assay, a chip-based expression platform, invader RNA assay platform or b-DNA detection platform.

10. The method of claim 8, wherein DEAR1 protein expression is evaluated using an ELISA, an immunoassay, a radioimmunoassay (RIA), an immunoradiometric assay, a fluoroimmunoassay, a chemiluminescent assay, a bioluminescent assay, a gel electrophoresis, a Western blot analysis, immunohistochemistry or a Luminex® protein assay.

11. The method of claim 10, wherein DEAR1 protein expression is evaluated using immunohistochemistry.

12. The method of claim 8, wherein DEAR1 protein expression is evaluated using an antibody that binds immunologically to a DEAR1 polypeptide.

13. The method of claim 12, wherein the antibody is a polyclonal antibody or a monoclonal antibody.

14. The method of claim 1, wherein determining if the cancer cell has a reduced level of DEAR1 expression and/or function comprises identifying the DEAR1 gene structure.

15. The method of claim 14, wherein a loss-of-function mutated gene structure of DEAR1 indicates a poor prognosis.

16. The method of claim 14, wherein the identifying comprises an assay selected from the group consisting of sequencing, wild-type oligonucleotide hybridization, mutant oligonucleotide hybridization, SSCP (single-strand conformation polymorphism), PCR and RNase protection.

17. The method of claim 14, wherein the identifying comprises determining loss of heterozygosity, mutations or DNA methylation.

18. The method of claim 15, wherein the loss-of-function mutated gene structure is a homologous or heterozygous deletion.

19. The method of claim 15, wherein the loss-of-function mutated gene structure results in loss of heterozygosity.

20. The method of claim 15, wherein the loss-of-function mutated gene structure comprises loss-of-function mutations.

21. The method of claim 15, wherein the loss-of-function mutated gene structure comprises a promoter mutation.

22. The method of claim 1, wherein the subject is undergoing cancer treatment.

23. The method of claim 22, wherein the cancer cell is obtained after the cancer treatment.

24. The method of claim 22, wherein the cancer cell is obtained during the cancer treatment.

25. The method of claim 22, wherein the cancer cell is obtained prior to the cancer treatment.

26. The method of claim 22, wherein the cancer treatment is surgery, radiotherapy, chemotherapy, and/or immunotherapy.

27. The method of claim 1, wherein the subject has a cancer selected from the group consisting of brain, lung, liver, spleen, kidney, lymph node, small intestine, pancreas, blood cells, colon, stomach, breast, endometrium, prostate, testicle, ovary, skin, head and neck, esophagus, bone marrow and blood cancer.

28. The method of claim 27, wherein the cancer is breast cancer.

29. The method of claim 28, wherein the breast cancer is an early onset breast cancer.

30. The method of claim 28, wherein the breast cancer is metastatic breast cancer.

31. The method of claim 1, wherein the cancer cell is obtained from a tissue sample, a surgical sample from tumor resection, a fluid sample, or a needle aspirate sample.

32. The method of claim 31, wherein the sample is a surgical sample from tumor resection.
